(19) United States
(12) Patent Application Publication
(10) Pub. No.: US 2010/0179168 A1
(43) Pub. Date: Jul. 15, 2010

(54) ARYL AND HETEROARYL SULPHONAMIDES AS GROWTH HORMONE SECRETAGOGUE RECEPTOR AGONISTS

(76) Inventors: Emma Louise Blaney, Essex (GB); David Kenneth Dean, Essex (GB); Alessandra Gaiba, Essex (GB); Nigel Paul King, Singapore (SG); Jason Witherington, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/296,965

(22) PCT Filed: Apr. 13, 2007
(86) PCT No.: PCT/EP07/53619

§ 371 Date: Oct. 13, 2008

(30) Foreign Application Priority Data
Apr. 13, 2006 (GB) 0607529.5
May 5, 2006 (GB) 0608978.3

(51) Int. Cl. A61K 31/497; C07D 307/36; C07D 405/12; C07D 333/22; A61K 31/341; A61K 31/381; A61K 31/443; A61P 1/00; A61P 1/04; A61P 1/06; A61P 9/00; A61P 19/10

(52) U.S. Cl. 514/254.1; 549/491; 544/379; 549/77; 546/283.4; 514/471; 514/438; 514/336

(57) ABSTRACT

The present invention therefore provides compounds of formula (I) or pharmaceutically acceptable salts thereof: (I) processes for their preparation, pharmaceutical compositions containing the same and to their use in the treatment of gastrointestinal and other disorders.

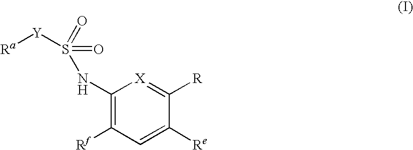

The present invention relates to novel compounds, processes for their preparation, pharmaceutical compositions containing the same and to their use in the treatment of gastrointestinal and other disorders.

Ghrelin is a 28 amino acid peptide predominantly produced by the stomach and to a lesser extent by the bowel, pancreas, kidney, placenta, pituitary and the arcuate nucleus of the hypothalamus. It has only recently been purified and isolated from the rat and human stomach (Kojima et al., Nature 1999; 402: 656), where it has been found in X/A endocrine cells associated with the acid-secreting parietal cells of the gastric glands. Studies have shown that ghrelin acts on growth hormone secretagogue receptors (GHS-R), stimulates the release of growth hormone, induces rat adiposity (Tschöp et al., Nature 2000, 407(6806), 908), controls gastric acid secretion (Masuda et al., Biochemical and Biophysical Research Communications 2000; 276: 905) and when released within the rodent arcuate nucleus (Kojima et al., Nature 1999; 402: 656; Lu et al., Neuroscience Letters. 2002; 321(3):157) or when administered i.c.v. (Nakazato et al., Nature 2001; 409: 194; Shintani et al., Diabetes 2001; 50: 227) stimulates an increase in food consumption. Systemically-administered ghrelin may also achieve the same, possibly by changing vagal nerve input to the brainstem vagal nuclei and hence, to the arcuate nucleus (Date et al., Gastroenterology 2002; 123: 1120). These studies indicate that GHS-R agonists have therapeutic utility in the treatment of different forms of cachexia and eating disorders.

Agonists of the ghrelin receptor have been described as useful in treating a growth hormone deficient state, stimulating an increase in food consumption thereby facilitating weight gain or maintenance of weight or appetite increase. This is particularly useful for a patient having a disease or disorder, or under going a treatment, that is accompanied by weight loss. Examples of diseases or disorders accompanied by weight loss include eating disorders (including anorexia, bulimia) cancer cachexia, AIDS, wasting, cachexia, and wasting in frail elderly. Examples of treatments accompanied by weight loss include chemotherapy, radiation therapy, temporary or permanent immobilization, and dialysis.

Further work with growth hormone secretagogues [e.g., WO 97/24369] suggests roles for ghrelin receptor agonists in the treatment or prevention of frailty associated with ageing, the acceleration of the repair of fractured bone, reducing protein catabolism after major surgery or during chronic illness, improving muscle strength and mobility control of congestive heart failure, and other metabolic disorders. Studies with such compounds also indicate a role in the promotion of sleep quality [WO 97/24369], and in the improvement of congestive heart failure after administration of ghrelin (Nagaya et al., J. Clin. Endocrinol. Metab. 2001, 86, 5854-5859; Circulation 2001, 104, 1430-1435).

In both anaesthetised and conscious rodents and in conscious dogs, ghrelin increases gastric motility and emptying (anaesthetised rat motility Masuda et al., Biochemical and Biophysical Research Communications 2000; 276: 905; rat gastric emptying Trudel et al., American Journal of Physiology 2002; 282: G948; mouse gastric emptying Asakawa et al., Gastroenterology 2001; 120: 337). This action can also be illustrated in vitro, by showing an ability of rat ghrelin to facilitate electrically-evoked, excitatory nerve-mediated contractions in rodent gastric fundus strips, a response mimicked by partial 5-HT₄ receptor agonists and indicative of a “prokinetic-like” response (Murray et al., British Journal of Pharmacology 2002; 136: 18P). Further, in conscious rats, i.c.v. administration of ghrelin reduces gastric acid secretion (Sibilia et al, Neuroendocrinology 2002; 75: 92); s.c. administration was without effect. Trudel and colleagues (American Journal of Physiology 2002; 282: G948) showed that ghrelin could reverse the gastric stasis created by invoking paralytic ileus via intestinal manipulation. Studies have shown that ghrelin increases gastric emptying in humans with diabetic gastroparesis (Murra et al, Gut 2005, 54, 1693), idiopathic gastroparesis (Tack et al, Aliment. Pharmacol. Ther., 2005, 22, 847) and neurogenic gastroparesis (Binn et al, Peptides 2006). Together, all of these data indicate that ghrelin might act as a gut hormone to facilitate both nutritional intake and digestion. This concurs with the proposal that the ability of ghrelin to evoke small reductions in pancreatic insulin secretion is consistent with the release of ghrelin during fasting conditions, when it will be important to maintain appropriate levels of blood sugars (see Muccioli et al., Eur J Pharmacology 2002, 440: 235).

Thus, in addition to conditions associated with cachexia (e.g. as a result of cancer), sarcopenia and/or those chronic diseases that may be exacerbated by loss of muscle mass (e.g. osteoporosis, rheumatoid arthritis, osteoarthritis, advancing age), growth hormone deficiency (e.g., when associated with age-related conditions), other disorders of metabolism, disorders in patterns of sleep or of congestive heart failure, GHS-R agonists will be useful treatments to alleviate symptoms associated with gastro-esophageal reflux and/or with dyspepsia, with or without appetite-/metabolic-related cachexia. Examples of such conditions include the reduction in feeding and the gastric stasis and emesis associated with anti-cancer treatment and other treatments or conditions which evoke similar symptoms, the gastroparesis associated with diabetes and gastroparesis and the symptoms associated with functional dyspepsia and gastro-esophageal reflux disease. Further, an ability to stimulate intestinal motility suggests that compounds active at ghrelin receptors will be useful treatments of paralytic ileus or pseudo-obstruction, and of conditions associated with constipation, such as constipation-predominant irritable bowel syndrome.

European patent application EP1159964 claims the use of compounds which stimulate the release of growth hormone as a means of stimulating the motility of the gastrointestinal system in a patient.

WO 95/06637 discloses a series of piperazine derivatives which are said to possess 5-HT_{1D} receptor antagonist activity. WO0236562, WO0132660, WO0005225, WO9942465 and WO9827081 all disclose arylpiperazine sulfonamide derivatives that are claimed to be 5-HT₆ receptor antagonists. WO0274764, WO0274768, and WO0123374 all disclose dimethylpiperazine derivatives that are claimed to be selective 5HT_{1B} receptor antagonists.

WO06/010629 discloses a series of arylpiperazine derivatives, which are said to possess agonistic activity at the growth hormone secretagogue (GHS) receptors.

We have now found a novel class of sulfonamide derivatives which exhibit a selective agonistic activity at the growth hormone secretagogue (GHS) receptors.

The present invention therefore provides compounds of formula (I) or pharmaceutically acceptable salts thereof:

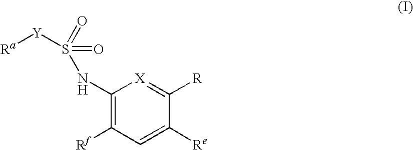

in which R^{a} is aryl or heteroaryl;
Y is a single bond, CH₂, CH₂CH₂, or CH═CH;

### X is CH or N;

R^{e} is hydrogen, C₁₋₆alkyl, C₃₋₆cycloalkyl, COC₁₋₆alkyl, C₁₋₆alkoxy, halogen, hydroxyl, trifluoromethyl, trifluoromethoxy or cyano;
R^{f} is hydrogen, C₁₋₆alkyl, C₃₋₆cycloalkyl, COC₁₋₆alkyl, C₁₋₆alkoxy, C₁₋₆alkoxyC₁₋₆alkyl, halogen, hydroxyl, trifluoromethyl, or cyano;
R is a group of formula (A):

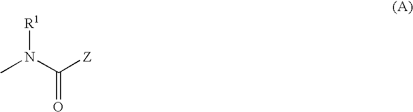

wherein R¹ is hydrogen or methyl;
Z is piperidine optionally substituted with methyl, cyclopentane substituted by amine or C(R²)(R³)N(R⁴)(R⁵);
R² and R³ are independently selected from hydrogen, methyl, ethyl, fluoromethyl and hydroxymethyl; and
R⁴ and R⁵ are independently selected from hydrogen, methyl, acetyl and N,N-dimethylaminomethylcarbonyl;
or R is a group of formula (B):

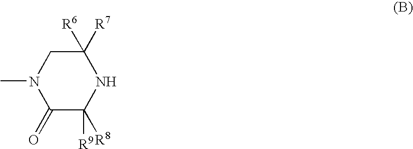

wherein R⁶⁻⁹ are independently selected from hydrogen and methyl and at least one of them is methyl.

Alkyl groups, whether alone or as part of another group, may be straight chain or branched. The term “halogen” is used herein to describe, unless otherwise stated, a group selected from fluorine, chlorine, bromine or iodine.

Suitable C₃₋₆cycloalkyl groups include cyclopropyl, cyclobutyl, cyclopentyl and cyclohexyl.

The term “aryl” as a group or part of a group includes phenyl and naphthyl. Where used herein the term naphthyl is intended, unless otherwise stated, to denote both naphth-1-yl and naphth-2-yl groups.

The term “heteroaryl” is intended to mean a 5-6 membered monocyclic aromatic or a fused 8-11 membered bicyclic aromatic ring containing heteroatoms selected from oxygen, nitrogen and sulphur.

When the term heteroaryl represents a 5 or 6 membered group it contains a heteroatom selected from O, N or S and may optionally contain a further 1 to 3 nitrogen atoms. When heteroaryl represents a 6-membered group it contains from 1 to 3 nitrogen atoms.

When the term heteroaryl represents a fused 8-11 membered bicyclic aromatic ring it contains 1 to 3 heteroatoms selected from O, N or S.

Suitable examples of such monocyclic aromatic rings include thienyl, furanyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. The term a fused 8-11 membered bicyclic aromatic group includes groups wherein one of the rings is partially saturated.

Suitable examples of such fused aromatic rings include benzofused heterocyclic rings such as quinolinyl, isoquinolinyl, quinazolinyl, quinoxalinyl, cinnolinyl, naphthyridinyl, indolyl, indazolyl, pyrrolopyridinyl, thienopyridyl, benzofuranyl, benzothienyl, benzimidazolyl, benzoxazolyl, benzisoxazolyl, benzothiazolyl, benzisothiazolyl, benzoxadiazolyl, benzothiadiazolyl, benzodioxanyl, indolinyl, isoindolinyl, tetrahydroquinolinyl, tetrahydroisoquinolinyl, benzazepinyl or chromanyl.

The aryl and heteroaryl groups according to the definitions above included such groups wherein they may be optionally substituted by one to three substituents which may be the same or different, and which are selected from the group consisting of halogen, hydroxy, cyano, nitro, oxo, trifluoromethyl, trifluoromethoxy, fluoromethoxy, difluoromethoxy, C₁₋₆ alkyl, C₃₋₆ cycloalkyl, C₁pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, aryloxy, heteroaryloxy, aroyl, aroylC₁₋₆ alkyl, arylC₁₋₆ alkanoyl, aryl, heteroaryl, heterocyclyl, or a group NR¹⁵R¹⁶, CONR¹⁵R¹⁶, SO₂NR NR¹⁵COR¹⁶ or NR¹⁵SO₂R¹⁶ wherein R¹⁵ and R¹⁶ independently represent hydrogen, C₁₋₆ alkyl, C₃₋₇ cycloalkyl, aryl, heteroaryl or together with the nitrogen atom to form a 5- to 7-membered non-aromatic heterocyclic ring which may optionally contain an additional ring member selected from O, S or NH.

When R^{a} is substituted by aryl or heteroaryl groups these substituents are optionally further substituted provided that the further substituents are not aryl or heteroaryl. Further substituents on such aryl and heteroaryl groups may for example be selected from halogen, cyano, C₁₋₆alkyl, C₁₋₆ alkoxy and oxo. Particularly chloro, cyano, methyl, and oxo. In another aspect, substituents on such aryl and heteroaryl groups may for example be selected from fluoro, methoxy and methoxymethyl

The term “heterocyclyl” is intended to mean a 4-7 membered monocyclic saturated or partially unsaturated aliphatic ring containing 1 to 3 heteroatoms selected from oxygen, sulphur or nitrogen. Suitable examples of such monocyclic rings include pyrrolidinyl, piperidinyl, piperazinyl, morpholinyl, thiomorpholinyl, diazepanyl, azepanyl, and tetrahydrofuranyl.

In a suitable group of compounds of formula (I):
R^{a} is aryl substituted by heteroaryl; and/or

### X is CH

Y is a single bond; and/or
R^{e} is hydrogen; and/or
R^{f} is alkoxy or hydrogen; and/or
R is a group of formula (A):

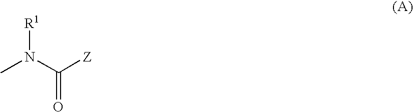

wherein
R¹ is hydrogen or methyl; and/or
Z is C(R²)(R³)N(R⁴)(R⁵); and/or
R² and R³ are independently selected from hydrogen, methyl, ethyl and hydroxymethyl; and/or
R⁴ and R⁵ are independently selected from hydrogen or methyl;
or R is a group of formula (B):

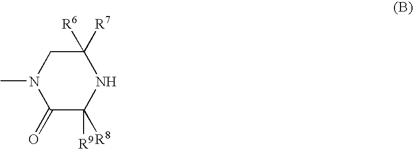

wherein R⁶ and R⁷ are hydrogen and R⁸ and R⁹ are methyl.
In another suitable group of compounds of formula (I):
R^{a} is phenyl substituted by methyl-furanyl; and/or

### X is CH or N

Y is a single bond; and/or
R^{e} is hydrogen; and/or
R^{f} is methoxy;
R is a group of formula (A):

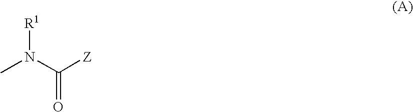

wherein
R¹ is hydrogen or methyl; and/or
Z is C(R²)(R³)N(R⁴)(R⁵); and/or
R² and R³ are independently selected from hydrogen and methyl; and/or
R⁴ and R⁵ are independently selected from hydrogen or methyl;
or R is a group of formula (B):

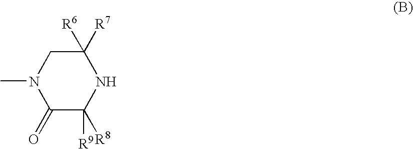

wherein R⁶ and R⁷ are hydrogen and R⁸ and R⁹ are methyl.

Specific examples of formula (I) are:
N¹-[3-({[3-Fluoro-4-(5-methyl-2-furanyl)phenyl]sulfonyl}amino)-4-(methyloxy)phenyl]-2-methylalaninamide
N-[5-(3,3-D methyl-2-oxo-1-piperazinyl)-2-(methyloxy)phenyl]-3-fluoro-4-(5-methyl-2-furanyl)benzenesulfonamide
N¹-[3-({[3-Fluoro-4-(5-methyl-2-furanyl)phenyl]sulfonyl}amino)-4-(methyloxy)phenyl]-D-alaninamide
N¹-[3-({[3-Fluoro-4-(5-methyl-2-furanyl)phenyl]sulfonyl}amino)-4-(methyloxy)phenyl]-L-alaninamide
N¹-[3-({[3-Fluoro-4-(5-methyl-2-furanyl)phenyl]sulfonyl}amino)-4-(methyloxy)phenyl]-glycinamide
N¹-[3-({[3-Fluoro-4-(5-methyl-2-furanyl)phenyl]sulfonyl}amino)-4-(methyloxy)phenyl]-N¹,2-dimethylalaninamide
(2S)-2-Amino-N-[3-({[3-fluoro-4-(5-methyl-2-furanyl)phenyl]sulfonyl}amino)-4-(methyloxy)phenyl]butanamide
N¹-[3-({[3-Fluoro-4-(5-methyl-2-furanyl)phenyl]sulfonyl}amino)-4-(methyloxy)phenyl]-L-serinamide
N¹-[3-({[3-Fluoro-4-(5-methyl-2-furanyl)phenyl]sulfonyl}amino)-4-(methyloxy)phenyl]-2-methylserinamide
N¹-[3-({[3-Fluoro-4-(5-methyl-2-furanyl)phenyl]sulfonyl}amino)-4-(methyloxy)phenyl]-N²-methyl-L-alaninamide
N¹-[3-({[3-Fluoro-4-(5-methyl-2-furanyl)phenyl]sulfonyl}amino)-4-(methyloxy)phenyl]-N²,2-dimethylataninamide
N¹-[3-({[2-Chloro-4-(5-methyl-2-furanyl)phenyl]sulfonyl}amino)-4-(methyloxy)phenyl]-2-methylalaninamide
2-Methyl-N¹-[3-({[4-(5-methyl-2-furanyl)phenyl]sulfonyl}amino)-4-(methyloxy)phenyl]alaninamide
N¹-[3-({[2-Chloro-4-(3-furanyl)phenyl]sulfonyl}amino)-4-(methyloxy)phenyl]-2-methylalaninamide
N¹-[3-({[3-Fluoro-4-(3-furanyl)phenyl]sulfonyl}amino)-4-(methyloxy)phenyl]-2-methylalaninamide
N¹-[3-({[2-Chloro-4-(5-methyl-2-furanyl)phenyl]sulfonyl}amino)-4-(methyloxy)phenyl]-N²-methyl-L-alaninamide
N¹-[3-({[4-(5-Methyl-2-furanyl)phenyl]sulfonyl}amino)-4-(methyloxy)phenyl]-D-alaninamide
N¹-[3-({[2-Chloro-4-(5-methyl-2-furanyl)phenyl]sulfonyl}amino)-4-(methyloxy)phenyl]-D-alaninamide
N¹-[3-({[2-Chloro-4-(5-methyl-2-furanyl)phenyl]sulfonyl}amino)-4-(methyloxy)phenyl]-L-alaninamide
N¹-[3-({[2-Chloro-4-(4-methyl-2-thienyl)phenyl]sulfonyl}amino)-4-(methyloxy)phenyl]-N²-methyl-L-alaninamide
N¹-[3-({[3-Chloro-3′-(methyloxy)-4-biphenylyl]sulfonyl}amino)-4-(methyloxy)phenyl]-N²-methyl-L-alaninamide
N¹-[3-({[2′-Fluoro-5′-(methyloxy)-4-biphenylyl]sulfonyl}amino)-4-(methyloxy)phenyl]-N²-methyl-L-alaninamide
N¹-[3-({[3-Chloro-2′-fluoro-5′-(methyloxy)-4-biphenylyl]sulfonyl}amino)-4-(methyloxy)phenyl]-N²-methyl-L-alaninamide
N¹-[3-({[2-Chloro-4-(5-methyl-2-furanyl)phenyl]sulfonyl}amino)-4-(methyloxy)phenyl]-N²-methyl-D-alaninamide
N¹-[5-({[3-Fluoro-4-(5-methyl-2-furanyl)phenyl]sulfonyl}amino)-2-(methyloxy)phenyl]-2-methylalaninamide
N¹-[3-({[3-Fluoro-4-(5-methyl-2-furanyl)phenyl]sulfonyl}amino)phenyl]-2-methylalaninamide
N¹-[4-Chloro-3-({[3-fluoro-4-(5-methyl-2-furanyl)phenyl]sulfonyl}amino)phenyl]-2-methylalaninamide
N¹-[4-Fluoro-3-({[3-fluoro-4-(5-methyl-2-furanyl)phenyl]sulfonyl}amino)phenyl]-2-methylalaninamide
N¹-[6-({[3-Fluoro-4-(5-methyl-2-furanyl)phenyl]sulfonyl}amino)-5-(methyloxy)-2-pyridinyl]-2-methylalaninamide

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolic or residue thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic, salicylic, lactic, mandelic or naphthalenesulfonic acid

The compounds of formula (I) may be prepared in crystalline or non-crystalline form, and, if crystalline, may optionally be hydrated or solvated. This invention includes within its scope stoichiometric hydrates as well as compounds containing variable amounts of water.

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

Compounds of the invention may be prepared using procedures which are analogous to those known in the art.

The compounds of formula (I) have been found to be GHS-R agonists in the GTPγS and FLIPR (Fluorometric Light Imaging Plate Reader) assay described herein.

Compounds of formula (I) and their pharmaceutically acceptable salts are therefore of use in the treatment of conditions or disorders which are mediated by compounds acting at the growth hormone secretagogue (GHS) receptors. In particular the compounds of formula (I) and their pharmaceutically acceptable salts are of use in the treatment of cachexia, sarcopenia, osteoporosis, rheumatoid arthritis, osteoarthritis, frailty associated with aging, growth hormone deficiency, metabolic disorders, sleep disorders, or congestive heart failure. The compounds of the invention will be useful treatments to alleviate symptoms associated with gastro-esophageal reflux and/or with dyspepsia, with or without appetite-/metabolic-related cachexia, the treatments of paralytic ileus or pseudo-obstruction, and of conditions associated with constipation, such as constipation-predominant irritable bowel syndrome.

It is to be understood that “treatment” as used herein includes prophylaxis as well as alleviation of established symptoms.

Thus the invention also provides a compound of formula (I) or a pharmaceutically acceptable salt thereof, for use as a therapeutic substance, in particular in the treatment of the conditions/disorders which can be mediated via the GHS receptors. In particular the invention provides a compound of formula (I) or a pharmaceutically acceptable salt thereof for use as a therapeutic substance in the treatment of cachexia, sarcopenia, osteoporosis, rheumatoid arthritis, osteoarthritis, frailty associated with aging, growth hormone deficiency, metabolic disorders, sleep disorders, congestive heart failure, alleviation of symptoms associated with gastro-esophageal reflux and/or with dyspepsia, with or without appetite-/metabolic-related cachexia, the treatments of paralytic ileus or pseudo-obstruction, and of conditions associated with constipation, such as constipation-predominant irritable bowel syndrome. It is to be understood that compounds of formula (I) may also be used in combination with other therapeutic substances.

The invention further provides a method of treatment of conditions or disorders in mammals including humans which can be mediated via the GHS receptors, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the GHS receptors.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months.

No toxicological effects are indicated/expected when a compound (of the invention) is administered in the above mentioned dosage range.

All publications, including but not limited to patents and patent applications, cited in this specification are herein incorporated by reference as if each individual publication were specifically and individually indicated to be incorporated by reference herein as though fully set forth.

The following descriptions and Examples illustrate the preparation of compounds of the invention. Each Example was characterised either as the free base or hydrochloride salt or occasionally as the formic acid salt directly from mass directed autoprep HPLC. The hydrochloride salts were prepared by dissolving the pure material in dichloromethane or methanol and acidifying with ethereal HCl.

Where so indicated in the experimental section microwave heating was performed in Biotage Initiator 60 or Personal Chemistry Optimiser instruments. These instruments allowed the control of temperature up to 250° C. and allowed pressures up to 20 bar with microwave radiation up to 300 W at 2.45 GHz.

### Conditions, Hardware and Software Used for Mass Directed Auto-Purification System

### Hardware

### Waters 2525 Binary Gradient Module

### Waters 515 Makeup Pump

### Waters Pump Control Module

### Waters 2767 Inject Collect

### Waters Column Fluidics Manager

### Waters 2996 Photodiode Array Detector

### Waters ZQ Mass Spectrometer

Gilson 202 fraction collector
Gilson Aspec waste collector

### Software

Waters Masslynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol:Water 80:20
Needle rinse solvent=Methanol

### Methods

There are four methods used depending on the analytical retention time of the compound of interest. They all have a 13.5-minute runtime, which comprises of a 10-minute gradient followed by a 13.5 minute column flush and re-equilibration step.

### Large/Small Scale 1.0-1.5=5-30% B

### Large/Small Scale 1.5-2.2=15-55% B

### Large/Small Scale 2.2-2.9=30-85% B

### Large/Small Scale 2.9-3.6=50-99% B

Large/Small Scale 3.6-5.0=80-99% B (in 6 mins)

### Flow Rate

All of the above methods have a flow rate of either 20 mLs/min (Small Scale) or 40 mLs/min (Large Scale)

### Conditions, Hardware and Software for Analytical LCMS Systems

### Hardware

### Agilent 1100 Gradient Pump

### Agilent 1100 Autosampler

### Agilent 1100 DAD Detector

### Agilent 1100 Degasser

### Agilent 1100 Oven

### Agilent 1100 Controller

### Waters ZQ Mass Spectrometer

### Sedere Sedex 55, Sedere Sedex 85 or Polymer Labs PL-ELS-2100

### Software

Waters MassLynx version 4.0 SP2

### Column

The column used is a Waters Atlantis, the dimensions of which are 4.6 mm×50 mm.

The stationary phase particle size is 3 μm.

### Solvents

A: Aqueous solvent=Water+0.05% Formic Acid
B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

### Flow Rate

The above method has a flow rate of 3 mL/min.

### Conditions Used for NMR

### Hardware

### Bruker 400 MHz Ultrashield

### Bruker B-ACS60 Autosampler

### Bruker Advance 400 Console

### Bruker DPX250

### Bruker AVANCE 500

### Bruker DRX600

### Software

User interface—NMR Kiosk
Controlling software—XWin NMR version 3.0

### EXAMPLES

### Description 1

### 4-Bromo-3-fluoro-N-[2-(methyloxy)-5-nitrophenyl]benzenesulfonamide (D1)

A solution of 2-(methyloxy)-5-nitroaniline (4.2 g, 25 mmol) in pyridine (30 mL) and dichloromethane (20 mL) was treated with a solution of 4-bromo-3-fluorobenzenesulfonyl chloride (8.2 g, 4.4 mL, 30 mmol) in dichloromethane (10 mL) and the reaction was stirred at room temperature for 30 minutes. The solvent was evaporated and the residue co-evaporated with toluene. The residue was partitioned between dichloromethane and saturated sodium bicarbonate solution. The organic layer was separated, washed with water and brine, dried and evaporated. The residue was triturated with ether and the solid was collected and dried to give the title product (D1). MS (ES−) m/c 403/405 [M−H]⁻.

### Description 2

### 3-Fluoro-4-(5-methyl-2-furanyl)-N-[2-(methyloxy)-5-nitrophenyl]benzenesulfonamide (D2)

A suspension of 4-bromo-3-fluoro-N-[2-(methyloxy)-5-nitrophenyl]benzenesulfonamide (D1) (7.3 g, 18 mmol) in 1,2-dimethoxyethane (200 mL) was stirred under argon at room temperature. A solution of sodium carbonate (9.5 g, 90 mmol) in water (100 mL) was added followed by (5-methyl-2-furanyl)boronic acid (4.54 g, 36 mmol) and bis(triphenylphosphine)palladium(II) chloride (25 mg, 0.036 mmol, 0.2 mol %). The reaction was heated at reflux for 1 hour. Two additional portions of (5-methyl-2-furanyl)boronic acid (1.2 g, 10 mmol) were added after 1 and 2 hours and a further portion of (5-methyl-2-furanyl)boronic acid (600 mg, 5 mmol) was added after 5 hours. After heating at reflux for a total of 6 hours the reaction mixture was cooled to room temperature and was diluted with ethyl acetate and water. The organic layer was separated, washed with water and brine, dried over anhydrous magnesium sulfate and evaporated. The residue was triturated with ether and the solid was filtered and dried to give the title product (D2). MS (ES−) m/e 405 [M−H]⁻.

### Description 3

### N-[5-Amino-2-(methyloxy)phenyl]-3-fluoro-4-(5-methyl-2-furanyl)benzenesulfonamide (D3)

A solution of 3-fluoro-4-(5-methyl-2-furanyl)-N-[2-(methyloxy)-5-nitrophenyl]benzenesulfonamide (D2) (4.0 g, 10 mmol) in tetrahydrofuran (100 mL) was treated with palladium on charcoal (10% paste, 200 mg) and the mixture was stirred under an atmosphere of hydrogen for 24 hours. The mixture was filtered through celite, washing with tetrahydrofuran and the filtrate was evaporated. The residue was triturated with ether/pentane and the solid was collected and dried to give the title product (D3). MS (ES+) m/e 377 [M+H]⁺.

### Description 4

### 1,1-Dimethylethyl (2-{[3-({[3-fluoro-4-(5-methyl-2-furanyl)phenyl]sulfonyl}amino)-4-(methyloxy)phenyl]amino}-1,1-dimethyl-2-oxoethyl)carbamate (D4)

A solution of N-[5-Amino-2-(methyloxy)phenyl]-3-fluoro-4-(5-methyl-2-furanyl)benzenesulfonamide (D3) (376 mg, 1 mmol) in dichloromethane (25 mL) was treated with N,N-diisopropylethylamine (258 mg, 0.35 mL, 2 mmol), N-{[(1,1-dimethylethyl)oxy]carbonyl}-2-methylalanine (223 mg, 1.1 mmol) and O-(benzotriazol-1-yl)-N,N—N′,N′-tetramethyluronium hexafluorophosphate (HBTU, 437 mg, 1.1 mmol). After stirring at room temperature for 22 hours the reaction mixture was partitioned between dichloromethane and saturated sodium bicarbonate solution. The organic layer was separated, washed with water and brine, dried and evaporated to give the title product (D4). MS (ES−) m/e 560 [M−H]⁻.

### Description 5

### 3,3-Dimethyl-2-piperazinone (D5)

To a stirred solution of 1,2-ethanediamine (7.5 g, 125 mmol) in toluene (10 mL) at 0-5° C. was added a solution of ethyl 2-bromo-2-methylpropanoate (4.87 g, 25 mmol) in toluene (5 mL) over 30 minutes. The reaction was stirred at room temperature for 1 hour and then heated at reflux for 22 hours. After cooling to room temperature the layers were separated, the bottom layer was extracted with toluene and the toluene extracts were combined and evaporated. The residue was purified by column chromatography (silica gel) eluting with dichloromethane/2M ammonia in methanol: (20:1 to 10:1) to afford the title product (D5). ¹HNMR (d6-DMSO): δ 7.36 (1H, b), 3.10 (2H, m), 2.81 (2H, m), 2.26 (1H, b), 1.16 (6H, s).

### Description 6

### 3,3-Dimethyl-1-[4-(methyloxy)-3-nitrophenyl]-2-piperazinone (D6)

A stirred mixture of 4-bromo-1-(methyloxy)-2-nitrobenzene (696 mg, 3 mmol), 3,3-dimethyl-2-piperazinone (D5) (460 mg, 3.6 mmol), potassium phosphate (1.27 g, 6 mmol), copper (I) iodide (57 mg, 0.3 mmol) and trans-1,2-cyclohexanediamine (69 mg, 0.6 mmol) in dioxane (18 mL) was heated at 140° C. for 3 hours in a microwave reactor. A solution of 0.880 ammonia (2 mL) and water (20 mL) was added and the mixture was extracted with ethyl acetate. The combined organic extracts were washed with water, brine, dried and evaporated. The residue was purified by column chromatography (silica gel) eluting with dichloromethane/2M ammonia in methanol: (20:1 to 10:1) to afford the title product (D6). MS (ES+) m/e 280 [M+H]⁺.

### Description 7

### 1-[3-Amino-4-(methyloxy)phenyl]-3,3-dimethyl-2-piperazinone (D7)

A solution of 3,3-dimethyl-1-[4-(methyloxy)-3-nitrophenyl]-2-piperazinone (D6) (160 mg, 0.57 mmol) in methanol (10 mL) was treated with palladium on charcoal (10% paste, 30 mg) and the mixture was stirred under an atmosphere of hydrogen for 21 hours. The mixture was filtered through celite, washing with methanol and the filtrate was evaporated to give the title product (D7). MS (ES+) m/e 250 [M+H]⁺.

### Description 8

### 4-Bromo-N-[5-(3,3-dimethyl-2-oxo-1-piperazinyl)-2-(methyloxy)phenyl]-3-fluorobenzenesulfonamide (D8)

The title compound (D8) was prepared from the product of Description 7 (D7) and 4-bromo-3-fluorobenzenesulfonyl chloride using a similar method to that described for Description 1 (D1). MS (ES+) 486/488 [M+H]⁺.

### Description D9

### 3-Fluoro-N-[5-(methylamino)-2-(methyloxy)phenyl]-4-(5-methyl-2-furanyl)benzenesulfonamide (D9)

A mixture of N-[5-amino-2-(methyloxy)phenyl]-3-fluoro-4-(5-methyl-2-furanyl)benzenesulfonamide (D3) (1.5 g, 4 mmol) in methanol (10 mL) and sodium methoxide (30% in methanol, 5 mL) was heated at reflux for 15 minutes. The suspension was cooled to 40° C. with continuous stirring and the resulting slurry was added to a stirred suspension of paraformaldehyde (240 mg, 8 mmol) in methanol (10 mL). The mixture was stirred at room temperature for 20 hours. Sodium borohydride (304 mg, 8 mmol) was added and the mixture was heated at reflux for 1 hour. The reaction mixture was cooled to room temperature and diluted with water and dichloromethane. The organic solvent was evaporated and the residue was partitioned between dichloromethane and water. The organic layer was separated, washed with water and brine, dried and evaporated. The residue was purified column chromatography (silica gel) eluting with 0-50% ethyl acetate in hexanes to give the title product (D9). MS (ES+) m/e 391 [M+H]⁺.

### Description D10

### 1,1-Dimethylethyl{2-[[3-({[3-fluoro-4-(5-methyl-2-furanyl)phenyl]sulfonyl}amino)-4-(methyloxy)phenyl](methyl)amino]-1,1-dimethyl-2-oxoethyl}carbamate (D10)

A solution of N-{[(1,1-dimethylethyl)oxy]carbonyl}-2-methylalanine (213 mg, 1.05 mmol) in N,N-dimethylformamide (1 mL) was treated with 3H-[1,2,3]triazolo[4,5-b]pyridin-3-ol (HOAt) (143 mg, 1.05 mmol) and N-[3-(dimethylamino)propyl]-N-ethylcarbodiimide hydrochloride (201 mg, 1.05 mmol) and the mixture was stirred at room temperature for 30 minutes. 3-Fluoro-N-[5-(methylamino)-2-(methyloxy)phenyl]-4-(5-methyl-2-furanyl)benzenesulfonamide (D9) (270 mg, 0.7 mmol) and N,N-diisopropylethylamine (270 mg, 0.37 mL, 2.1 mmol) were added and the reaction mixture was stirred at room temperature under argon for 72 hours. The mixture was diluted with dichloromethane and the solution was washed with saturated sodium hydrogen carbonate solution, water and brine, dried and evaporated. The residue was purified by silica gel chromatography eluting with 0-100% ethyl acetate in hexanes to give the title product (D10). MS (ES+) m/e 576 [M+H]⁺.

### Description D11

### N-[5-Amino-2-(methyloxy)phenyl]-3-fluoro-4-(5-methyl-2-furanyl)benzenesulfonamide (D11)

A solution of 3-fluoro-4-(5-methyl-2-furanyl)-N-[2-(methyloxy)-5-nitrophenyl]benzenesulfonamide (D2) (2.65 g, 6.5 mmol) in tetrahydrofuran (50 mL) was treated with palladium on charcoal (10% paste, 100 mg) and the mixture was stirred under an atmosphere of hydrogen for 18 hours. The mixture was filtered through celite, washing with tetrahydrofuran and the filtrate was evaporated. As it still contained starting material, the mixture was dissolved in tetrahydrofuran (50 mL), treated with palladium on charcoal (10% paste, 150 mg) and the mixture was stirred under an atmosphere of hydrogen overnight then for a further 7 hours. Additional palladium on charcoal (10% paste, 150 mg) was added and the mixture was stirred under an atmosphere of hydrogen overnight. The mixture was filtered through celite, washing with tetrahydrofuran and the filtrate was evaporated. The residue was triturated with methanol to give the title product. The filtrate was evaporated in vacuo, filtered through celite and triturated with ether/pentane 1:4 to afford another crop of the title compound (D11). MS (ES+) m/e 377 [M+H]⁺.

### Description 12

### 1,1-Dimethylethyl [(1S)-1-({[3-({[3-fluoro-4-(5-methyl-2-furanyl)phenyl]sulfonyl}amino)-4-(methyloxy)phenyl]amino}carbonyl)propyl]carbamate (D12)

A solution of N-[5-amino-2-(methyloxy)phenyl]-3-fluoro-4-(5-methyl-2-furanyl)benzenesulfonamide (D11) (110 mg, 0.29 mmol) in dichloromethane (3 mL) was treated with N,N-diisopropylethylamine (100 ul, 0.58 mmol), (2S)-2-({[(1,1-dimethylethyl)oxy]carbonyl}amino)butanoic acid (71 mg, 0.35 mmol) and O-(benzotriazol-1-yl)-N,N—N′,N′-tetramethyluronium hexafluorophosphate (HBTU, 132 mg, 0.35 mmol). After stirring at room temperature overnight the reaction mixture was partitioned between dichloromethane and saturated sodium bicarbonate solution. The organic layer was separated, washed with water and brine, dried over magnesium sulfate and evaporated to give the title product (D12). MS (ES−) m/e 560 [M−H]⁻.

### Description 13

### 1,1-Dimethylethyl [(1S)-2-{[3-({[3-fluoro-4-(5-methyl-2-furanyl)phenyl]sulfonyl}amino)-4-(methyloxy)phenyl]amino}-1-(hydroxymethyl)-2-oxoethyl]carbamate (D13)

The N²-{[(1,1-dimethylethyl)oxy]carbonyl}-L-serinamide (1.08 mmol, 0.221 g), N-[5-amino-2-(methyloxy)phenyl]-3-fluoro-4-(5-methyl-2-furanyl)benzenesulfonamide (D3) (0.54 mmol, 0.2 g), O-(7-azabenzotriazole-1-yl)-N,N,N′,N′-tetramethyluronium hexafluorophosphate (HATU, 1.08 mmol, 0.41 g), N-hydroxybenzotriazole (HOBt, 0.54 mmol, 0.073 g) and N,N-diisopropylethylamine (1.62 mmol, 0.28 mL) were added to N,N-dimethylformamide (10 mL) and stirred at room temperature under argon for 3 hours. The solvent was removed in vacuo and the resultant oil was dissolved in dichloromethane and washed with saturated sodium hydrogen carbonate solution and brine, dried over sodium sulfate and concentrated. The residue was purified column chromatography (silica gel) eluting with 0-100% ethyl acetate in pentane to give the title product (D13). MS (ES+) m/e 564 [M+H]⁺.

### Description 14

### N-{[(1,1-Dimethylethyl)oxy]carbonyl}-2-methylserine (D14)

α-methyl serine (650 mg, 5.46 mmol) was suspended in dichloromethane (15 mL) and the N,O-bis(trimethylsilyl)trifluoroacetamide (2.7 mL, 10.92 mmol) was added to the reaction mixture. This mixture was heated at reflux for 1 hour to give a homogenous solution, which was cooled before di-tert-butyl dicarbonate (1.25 g, 5.7 mmol) in dichloromethane (5 mL) was added portionwise. The mixture was left to stir at room temperature overnight. It was then washed with water and the organic layer dried over sodium sulfate, then concentrated in vacuo. This residue was redissolved in methanol and heated at 40° C. under argon for 3 hours. The solvent was removed in vacuo and re-evaporated from toluene (×3) to give the title product (D14). MS (ES−) m/e 218 [M−H]⁻.

### Description 15

### 1,1-Dimethylethyl [2-{[3-({[3-fluoro-4-(5-methyl-2-furanyl)phenyl]sulfonyl}amino)-4-(methyloxy)phenyl]amino}-1-(hydroxymethyl)-1-methyl-2-oxoethyl]carbamate (D15)

The title compound (D15) was prepared from the product of Description 14 (D14) and the product of Description 3 (D3) in a similar method to that described for Description 13 (D13). MS (ES−) m/e 576 [M−H]⁻.

### Description 16

### 1,1-Dimethylethyl ((1S)-2-{[3-({[3-fluoro-4-(5-methyl-2-furanyl)phenyl]sulfonyl}amino)-4-(methyloxy)phenyl]amino}-1-methyl-2-oxoethyl)methylcarbamate (D16)

A solution of N-[5-amino-2-(methyloxy)phenyl]-3-fluoro-4-(5-methyl-2-furanyl)benzenesulfonamide (D11) (150 mg, 0.40 mmol) in dichloromethane (3 mL) was treated with N,N-diisopropylethylamine (140 ul, 0.8 mmol), N-{[(1,1-dimethylethyl)oxy]carbonyl}-N-methyl-L-alanine (97 mg, 0.48 mmol) and O-(benzotriazol-1-yl)-N,N—N′,N′-tetramethyluronium hexafluorophosphate (HBTU, 182 mg, 0.48 mmol). After stirring at room temperature overnight the reaction mixture was diluted with methanol and purified by SCX cartridge eluting with methanol. The fractions were combined and evaporated in vacuo, the residue further purified by column chromatography (silica gel) eluting with 10-50% ethyl acetate in hexanes to give the title product (D16). MS (ES+) m/e 562 [M+H]⁺.

### Description 17

### 4-Bromo-3-fluoro-N-[2-(methyloxy)-5-nitrophenyl]benzenesulfonamide (D17)

A solution of 2-(methyloxy)-5-nitroaniline (3.0 g, 17.8 mmol) in pyridine (15 mL) and dichloromethane (15 mL) was cooled to 0° C. and treated with a solution of 4-bromo-3-fluorobenzenesulfonyl chloride (5.3 g, 20 mmol) in dichloromethane (5 mL) and the reaction was stirred at 0° C. to room temperature for 2 hours. The solvent was evaporated and the residue co-evaporated with toluene. The residue was triturated with methanol to give the title product. MS (ES−) m/e 403/405 [M−H]⁻.

### Description 18

### 3-Fluoro-4-(5-methyl-2-furanyl)-N-[2-(methyloxy)-5-nitrophenyl]benzenesulfonamide (D18)

A solution of 4-bromo-3-fluoro-N-[2-(methyloxy)-5-nitrophenyl]benzenesulfonamide (D17) (5.0 g, 12.3 mmol) in 1,2-dimethoxyethane (50 mL) was treated with a solution of sodium carbonate (5.2 g, 50 mmol) in water (52.5 mL). Bis(triphenylphosphine)palladium(II) chloride (86 mg, 0.12 mmol, 1 mol %) was added, the reaction was heated at 75° C. then treated with 4,4,5,5-tetramethyl-2-(5-methyl-2-furanyl)-1,3,2-dioxaborolane (5.1 g, 24.7 mmol) and the mixture heated at 75° C. overnight. The mixture was then diluted with ethyl acetate and water. The layers were separated and the aqueous layer extracted into ethyl acetate (×2). The organic layers were combined, washed with water and brine, dried over magnesium sulfate and evaporated. The residue was triturated with ether then methanol to give the title product (D18). MS (ES+) m/e 407 [M+H]⁺.

### Description 19

### N-[5-Amino-2-(methyloxy)phenyl]-3-fluoro-4-(5-methyl-2-furanyl)benzenesulfonamide (D19)

A solution of 3-fluoro-4-(5-methyl-2-furanyl)-N-[2-(methyloxy)-5-nitrophenyl]benzenesulfonamide (D18) (2.2 g, 5.4 mmol) in tetrahydrofuran (50 mL) was treated with palladium on charcoal (10% paste, 100 mg) and the mixture was stirred under an atmosphere of hydrogen for 18 hours. The mixture was filtered through celite, washing with tetrahydrofuran and the filtrate was evaporated. The residue was triturated with ether/pentane (1:4) and the solid was collected and dried to give the title product (D19). MS (ES+) m/e 377 [M+H]⁺.

### Description 20

### 4-Bromo-2-chloro-N-[2-(methyloxy)-5-nitrophenyl]benzenesulfonamide (D20)

4-Bromo-2-chlorobenzenesulfonyl chloride (6.96 g, 24.0 mmol) in dichloromethane (50 mL) was added dropwise with stirring to a solution of 2-(methyloxy)-5-nitroaniline (3.36 g, 20.0 mmol) and pyridine (2.37 g, 30.0 mmol) in dichloromethane (25 mL). The reaction mixture was stirred overnight at room temperature and then filtered to afford a batch of the title compound (D20). MS (ES−) m/e 419/421/423 [M−H]⁻. The filtrate was evaporated to dryness and the resulting solid was treated with dichloromethane. The insoluble material was filtered to afford a second batch of the title compound (D20). MS (ES−) m/e 419/421/423 [M−H]⁻.

### Description 21

### N-[5-Amino-2-(methyloxy)phenyl]-4-bromo-2-chlorobenzenesulfonamide (D21)

4-Bromo-2-chloro-N-[2-(methyloxy)-5-nitrophenyl]benzenesulfonamide (D20) (6.21 g, 14.7 mmol) in methanol (75 mL) was treated with ammonium formate (13.06 g, 210 mmol) and 5% Pt/C paste (57% water) (4.0 g) was added under argon. The reaction mixture was stirred at room temperature under argon. After 16 hours further 5% Pt/C paste (57% water) (2.0 g) was added and the mixture was allowed to stir at room temperature for a further 24 hours. The reaction mixture was filtered, the filtrate was evaporated to dryness and the residue redissolved in dichloromethane (75 mL) and washed with saturated sodium hydrogen carbonate solution (3×50 mL) and brine (1×50 mL). The solution was dried over magnesium sulfate, filtered and evaporated to give the title compound (D21). MS (ES+) m/e 391/393/394 [M+H]⁺. The filtered catalyst was extracted with ethyl acetate and the extracts were washed with brine, dried over magnesium sulfate, and then evaporated to dryness to give a second batch of the title compound (D21). MS (ES+) 391/393/394 [M+H]⁺.

### Description 22

### N¹-[3-{[(4-Bromo-2-chlorophenyl)sulfonyl]amino}-4-(methyloxy)phenyl]-2-methylalaninamide (D22)

The N-[5-amino-2-(methyloxy)phenyl]-4-bromo-2-chlorobenzenesulfonamide (0.56 mmol, 0.2 g) (D21) and 2-methylalanyl chloride¹ (0.784 mmol, 0.095 g) were suspended in dichloromethane (5 mL) under argon and the pyridine (0.84 mmol, 68 uL) was added dropwise. This mixture was stirred at room temperature overnight under argon. A further portion of 2-methylalanyl chloride (0.784 mmol, 0.095 g) and pyridine (0.84 mmol, 68 uL) was added and stirred overnight. The reaction mixture was washed with saturated sodium hydrogen carbonate solution and the dichloromethane layer was removed using a Phase-Sep cartridge. The organic layer was concentrated and the residue was purified by column chromatography (silica gel) eluting with 0-10% methanol/dichloromethane. The relevant fractions were combined and concentrated to give the title compound (D22). MS (ES+) m/e 478/480 [M+H]⁺.
1. Stavinoha, Jerome L.; Mariano, Patrick S.; Leone-Bay, Andrea; Swanson, Rosemarie; Bracken, Christopher. Journal of the American Chemical Society (1981), 103(11), 3148-60.

### Description 23

### 4-Bromo-N-[2-(methyloxy)-5-nitrophenyl]benzenesulfonamide (D23)

4-Bromobenzenesulfonyl chloride (6.13 g, 24.0 mmol) in dichloromethane (35 mL) was added dropwise with stirring to a solution of 2-(methyloxy)-5-nitroaniline (3.36 g, 20.0 mmol) and pyridine (2.37 g, 30.0 mmol) in dichloromethane (65 mL). The reaction mixture was stirred overnight at room temperature and then washed with dil. HCl (3×50 mL) followed by brine (1×30 mL). It was dried over magnesium sulfate and then evaporated to dryness. This was washed with hexane and the resulting solid was dried in vacuo to afford the title compound (D23). MS (ES−) m/e 385/387 [M−H]⁻.

### Description 24

### N-[5-Amino-2-(methyloxy)phenyl]-4-bromobenzenesulfonamide (D24)

4-Bromo-N-[2-(methyloxy)-5-nitrophenyl]benzenesulfonamide (D23) (6.70 g, 17.3 mmol) in methanol (75 mL) was treated with ammonium formate (15.32 g, 240 mmol) and 5% Pt/C paste (57% water) (4.0 g) was added under argon. The reaction mixture was stirred at room temperature under argon. After 21 hours, further 5% Pt/C (dry powder) (2.0 g) was added and the reaction mixture was stirred for a further 4 days. The reaction mixture was filtered and the residue was washed with methanol and ethyl acetate. The combined filtrate was evaporated to dryness and the residue redissolved in dichloromethane (50 mL) and washed with sodium hydrogen carbonate solution (3×30 mL) and brine (1×30 mL). The solution was dried over magnesium sulfate, filtered and evaporated. The resulting solid was washed with toluene (100 mL) and dried in vacuo to give the title compound (D24). MS (ES+) m/e 357/359 [M+H]⁺.

### Description 25

### N¹-[3-{[(4-bromophenyl)sulfonyl]amino}-4-(methyloxy)phenyl]-2-methylalaninamide (D25)

The title compound (D25) was prepared from the product of Description 24 (D24) and 2-methylalanyl chloride¹ using a similar method to that described for Description 22 (D22). MS (ES+) m/e 442/444 [M+H]⁺.

### Description 26

### 4-Bromo-3-fluoro-N-[2-(methyloxy)-5-nitrophenyl]benzenesulfonamide (D26)

4-Bromo-3-fluorobenzenesulfonyl chloride (13.12 g, 48.0 mmol) in dichloromethane (40 mL) was added dropwise with stirring to a solution of 2-(methyloxy)-5-nitroaniline (6.72 g, 40.0 mmol) and pyridine (2.37 g, 30.0 mmol) in dichloromethane (60 mL). The reaction mixture was stirred overnight at room temperature and then filtered to afford a batch of the title compound (D26). MS (ES−) m/e 403/405 [M−H]⁻ The filtrate from above was washed with dil. HCl (3×50 mL) followed by brine (1×30 mL). It was dried over magnesium sulfate and then evaporated to dryness. This was washed with hexane and the resulting solid was dried in vacuo to afford the title compound (D26). MS (ES−) m/e 403/405 [M−H]⁻.

### Description 27

### N-[5-Amino-2-(methyloxy)phenyl]-4-bromo-3-fluorobenzenesulfonamide (D27)

4-Bromo-3-fluoro-N-[2-(methyloxy)-5-nitrophenyl]benzenesulfonamide (D26) (15.68 g, 38.7 mmol) in methanol (120 mL) was treated with ammonium formate (34.4 g, 550 mmol) and 5% Pt/C paste (57% water) (8.0 g) was added under argon. The reaction mixture was stirred at room temperature under argon. After 16 hours further, 5% Pt/C paste (57% water) (4.0 g) was added, followed 3.5 hours later with 5% Pt/C (dry powder) (2.5 g). The mixture was then allowed to stir at room temperature for a further 4 days. The reaction mixture was filtered and the residue was washed with methanol and ethyl acetate. The combined filtrate was evaporated to dryness and the residue redissolved in dichloromethane (100 mL) and washed with sodium hydrogen carbonate solution (3×50 mL) and brine (1×50 mL). The solution was dried over magnesium sulfate, filtered and evaporated. This was washed with toluene (100 mL) and dried in vacuo to give the title compound (D27). MS (ES+) m/e 375/377 [M+H]⁺. The filtered catalyst was extracted with further ethyl acetate and the extracts were evaporated to dryness to leave a solid which was virtually identical to the earlier batch of the title compound. MS (ES+) m/e 375/377 [M+H]⁺ and 416/418 [M+H+CH₃CH₃CN]⁺.

### Description 28

### N¹-[3-{[(4-Bromo-3-fluorophenyl)sulfonyl]amino}-4-(methyloxy)phenyl]-2-methylalaninamide (D28)

The title compound (D28) was prepared from the product of Description 27 (D27) and 2-methylalanyl chloride¹ using a similar method to that described for Description 22 (D22). MS (ES+) m/e 440/442 [M+H]⁺.

### Description 29

### 1,1-Dimethylethyl ((1S)-2-{[3-{[(4-bromo-2-chlorophenyl)sulfonyl]amino}-4-(methyloxy)phenyl]amino}-1-methyl-2-oxoethyl)methylcarbamate (D29)

N-{[(1,1-Dimethylethyl)oxy]carbonyl}-N-methyl-L-alanine (0.207 g, 1.022 mmol) was dissolved in N,N-dimethylformamide (3 mL) and N-hydroxybenzotriazole (HOBt, 0.137 g, 1.022 mmol), diisopropylethylamine (0.177 mL, 1.022 mmol) and N-[3-(dimethylamino)propyl]N′-ethylcarbodiimide hydrochloride (0.195 g, 1.022 mmol) added and the reaction stirred at room temperature for 20 minutes. N-[5-amino-2-(methyloxy)phenyl]-4-bromo-2-chlorobenzenesulfonamide (0.20 g, 0.511 mmol) was then added in one portion and the reaction stirred at room temperature overnight. The reaction mixture was evaporated to a minimum. The crude product was dissolved in diethyl ether (50 mL) and saturated aqueous sodium hydrogen carbonate (30 mL). The organic layer was washed with further saturated aqueous sodium hydrogen carbonate (2×30 mL) and brine (30 mL). The organic layer was dried over magnesium sulfate to give the title product (D29). MS (ES+) m/e 576/578 [M+H]⁺.

### Description 30

### 1,1-Dimethylethyl ((1S)-2-{[3-({[2-chloro-4-(5-methyl-2-furanyl)phenyl]sulfonyl}amino)-4-(methyloxy)phenyl]amino}-1-methyl-2-oxoethyl)methylcarbamate (D30)

1,1-Dimethylethyl ((1S)-2-{[3-{[(4-bromo-2-chlorophenyl)sulfonyl]amino}-4-(methyloxy)phenyl]amino}-1-methyl-2-oxoethyl)methylcarbamate (D29) (0.1 g, 0.173 mmol), 4,4,5,5-tetramethyl-2-(5-methyl-2-furanyl)-1,3,2-dioxaborolane (0.057 g, 0.276 mmol), dichlorobis(triphenylphosphine)palladium (II) (0.0064 g, 0.009 mmol) and sodium carbonate (0.070 g, 0.738 mmol) in 1,2-dimethoxyethane (2 mL)/water (1 mL), were heated at 120° C. for 20 minutes in the microwave reactor. The reaction mixture was then dissolved in diethyl ether (20 mL) and washed with saturated aqueous sodium hydrogen carbonate (2×15 mL) and brine (15 mL). The organic layer was dried over magnesium sulfate and evaporated. It was then purified by chromatography (silica gel) eluting with 0 to 100% ethyl acetate/pentane to give the title product (D30). MS (ES+) m/e 578/580 [M+H]⁺.

### Description 31

### 1,1-Dimethylethyl ((1R)-2-{[3-{[(4-bromophenyl)sulfonyl]amino}-4-(methyloxy)phenyl]amino}-1-methyl-2-oxoethyl)carbamate (D31)

The title compound (D31) was prepared from the product of Description 24 (D24) and N-{[(1,1-dimethylethyl)oxy]carbonyl}-D-alanine using a similar method to that described for Description 29. MS (ES+) 526/528 [M+H]⁺.

### Description 32

### 1,1-Dimethylethyl ((1R)-1-methyl-2-{[3-({[4-(5-methyl-2-furanyl)phenyl]sulfonyl}amino)-4-(methyloxy)phenyl]amino}-2-oxoethyl)carbamate (D32)

The title compound (D32) was prepared from the product of Description 31 (D31) and 4,4,5,5-tetramethyl-2-(5-methyl-2-furanyl)-1,3,2-dioxaborolane using a similar method to that described for Description 30. MS (ES+) 530 [M+H]⁺.

### Description 33

### 1,1-Dimethyl-ethyl ((1R)-2-{[3-{[(4-bromo-2-chlorophenyl)sulfonyl]amino}-4-(methyloxy)phenyl]amino}-1-methyl-2-oxoethyl)carbamate (D33)

The title compound (D33) was prepared from the product of Description 21 (D21) and N-{[(1,1-dimethylethyl)oxy]carbonyl}-D-alanine using a similar method to that described for Description 29. MS (ES+) 562/564/566 [M+H]⁺.

### Description 34

### 1,1-Dimethylethyl ((1R)-2-{[3-({[2-chloro-4-(5-methyl-2-furanyl)phenyl]sulfonyl}amino)-4-(methyloxy)phenyl]amino}-1-methyl-2-oxoethyl)carbamate (D34)

The title compound (D34) was prepared from the product of Description 33 (D33) and 4,4,5,5-tetramethyl-2-(5-methyl-2-furanyl)-1,3,2-dioxaborolane using a similar method to that described for Description 30. MS (ES+) 564/566 [M+H]⁺.

### Description 35

### 1,1-Dimethylethyl ((1S)-2-{[3-{[(4-bromo-2-chlorophenyl)sulfonyl]amino}-4-(methyloxy)phenyl]amino}-1-methyl-2-oxoethyl)carbamate (D35)

N-{[(1,1-Dimethylethyl)oxy]carbonyl}-L-alanine (211 mg, 1.12 mmol), N-[3-(dimethylamino)propyl]-N′-ethylcarbodiimide hydrochloride (215 mg, 1.12 mmol) and N-hydroxybenzotriazole (171 mg, 1.12 mmol) in N,N-dimethylformamide (3 mL) were stirred together at room temperature for 15 minutes. N-[5-Amino-2-(methyloxy)phenyl]-4-bromo-2-chlorobenzenesulfonamide (D21) (220 mg, 0.56 mmol) was then added and the reaction mixture was stirred at room temperature overnight. It was then evaporated to dryness and the residue was partitioned between diethyl ether (30 mL) and dil. HCl (10 mL). The organic layer was separated and washed with sodium hydrogen carbonate solution (2×10 mL) and brine (1×10 mL). It was dried and evaporated to leave the title compound (D35). MS (ES+) 562/564/566 [M+H]⁺.

### Description 36

### 1,1-Dimethylethyl ((1S)-2-{[3-({[2-chloro-4-(5-methyl-2-furanyl)phenyl]sulfonyl}amino)-4-(methyloxy)phenyl]amino}-1-methyl-2-oxoethyl)carbamate (D36)

The title compound (D36) was prepared from the product of Description 35 (D35) and 4,4,5,5-tetramethyl-2-(5-methyl-2-furanyl)-1,3,2-dioxaborolane using a similar method to that described for Description 30. MS (ES+) 564/566 [M+H]⁺ and 586/588 [M+Na]⁺.

### Description 37

### 1,1-Dimethylethyl ((1S)-2-{[3-({[2-chloro-4-(4-methyl-2-thienyl)phenyl]sulfonyl}amino)-4-(methyloxy)phenyl]amino}-1-methyl-2-oxoethyl)methylcarbamate (D37)

1,1-Dimethylethyl ((1S)-2-{[3-{[(4-bromo-2-chlorophenyl)sulfonyl]amino}-4-(methyloxy)phenyl]amino}-1-methyl-2-oxoethyl)methylcarbamate (D29) (0.1 g, 0.173 mmol), (4-methyl-2-thienyl)boronic acid (0.036 g, 0.276 mmol), dichloro-bis(triphenylphosphine)palladium (11) (0.0064 g, 0.009 mmol) and sodium carbonate (35 mg, 0.369 mM) in 1,2-dimethoxyethane (2 mL)/water (1 mL), were heated at 120° C. for 20 minutes in the microwave reactor. The reaction mixture was then dissolved in diethyl ether (20 mL) and washed with saturated aqueous sodium hydrogen carbonate (2×15 mL) and brine (15 mL). The organic layer was dried over magnesium sulfate and evaporated and then purified by chromatography (silica gel), eluting with 0 to 100% ethyl acetate/pentane. The product fractions were evaporated to give the title compound (D37). MS (ES+) m/e 594/596 [M+H]⁺.

### Description 38

### 1,1-Dimethylethyl ((1S)-2-{[3-{[(2-chlorophenyl)sulfonyl]amino}-4-(methyloxy)phenyl]amino}-1-methyl-2-oxoethyl)methylcarbamate (D38)

N-{[(1,1-Dimethylethyl)oxy]carbonyl}-N-methyl-L-alanine (0.98 g, 5.34 mmol) was dissolved in N,N-dimethylformamide (20 mL) and N-hydroxybenzotriazole (0.69 g, 5.12 mmol), diisopropylethylamine (0.89 mL, 5.34 mmol) and N-[3-(dimethylamino)propyl]-N-ethylcarbodiimide hydrochloride (1.02 g, 5.34 mmol) added and the reaction stirred at room temperature for 20 minutes. N-[5-amino-2-(methyloxy)phenyl]-4-bromo-2-chlorobenzenesulfonamide (D21) (1 g, 2.56 mmol) was then added in one portion and the reaction stirred at room temperature overnight. The reaction mixture was evaporated to a minimum. The crude product was dissolved in diethyl ether and saturated aqueous sodium hydrogen carbonate. The organic layer was washed with further saturated aqueous sodium hydrogen carbonate and brine. The organic layer was dried over sodium sulfate to give the title compound (D38). MS (ES+) m/e 574/576 [M+H]⁺.

### Description 39

### 1,1-Dimethylethyl ((1S)-2-{[3-({[3-chloro-3′-(methyloxy)-4-biphenylyl]sulfonyl}amino)-4-(methyloxy)phenyl]amino}-1-methyl-2-oxoethyl)methylcarbamate (D39)

1,1-Dimethylethyl ((1S)-2-{[3-{[(2-chlorophenyl)sulfonyl]amino}-4-(methyloxy)phenyl]amino}-1-methyl-2-oxoethyl)methylcarbamate (D38) (0.2 g, 0.35 mmol), 3-(methyloxy)phenyl]boronic acid (0.071 g, 0.53 mmol), dichloro-bis(triphenylphosphine)palladium (II) (0.012 g, 0.017 mmol) and sodium carbonate (148 mg, 1.4 mmol) in 1,2-dimethoxyethane (2 mL)/water (1 mL), were heated at 120° C. for 20 minutes in the microwave reactor. The reaction mixture was then dissolved in diethyl ether and washed with saturated aqueous sodium hydrogen carbonate and brine. The organic layer was dried over magnesium sulfate and evaporated and then purified by chromatography (silica gel), eluting with 0 to 50% ethyl acetate/pentane. The product fractions were evaporated to give the title compound (D39). MS (ES+) m/e 604 [M+H]⁺.

### Description 40

### 1,1-Dimethylethyl ((1S)-2-{[3-{[(4-bromophenyl)sulfonyl]amino}-4-(methyloxy)phenyl]amino}-1-methyl-2-oxoethyl)methylcarbamate (D40)

N-{[(1,1-Dimethylethyl)oxy]carbonyl}-N-methyl-L-alanine (1.14 g, 5.6 mmol) was dissolved in N,N-dimethylformamide (17 mL) and N-hydroxybenzotriazole (1.07 g, 5.36 mmol), diisopropylethylamine (0.97 mL, 5.36 mmol) and N-[3-(dimethylamino)propyl]-N-ethylcarbodiimide hydrochloride (1.02 g, 5.34 mmol) added and the reaction stirred at room temperature for 20 minutes. N-[5-amino-2-(methyloxy)phenyl]-4-bromobenzenesulfonamide (D24) (1 g, 2.8 mmol) was then added in one portion and the reaction stirred at room temperature overnight. The reaction mixture was evaporated to a minimum and the crude product dissolved in diethyl ether (50 mL) and saturated aqueous sodium hydrogen carbonate (30 mL). The organic layer was washed with further saturated aqueous sodium hydrogen carbonate (2×30 mL) and brine (30 mL). The organic layer was dried over magnesium sulfate to give the title compound (D40). MS (ES+) m/e 542/544 [M+H]⁺.

### Description 41

### 1,1-Dimethylethyl ((1S)-2-{[3-({[2′-fluoro-5-(methyloxy)-4-biphenylyl]sulfonyl}amino)-4-(methyloxy)phenyl]amino}-1-methyl-2-oxoethyl)methylcarbamate (D41)

The title compound (D41) was prepared from the product of Description 40 (D40) and 2-fluoro-5-(methoxy)phenyl]boronic acid using a similar method to that described for Description 39. MS (ES+) m/e 588 [M+H]⁺.

### Description 42

### 1,1-Dimethylethyl ((1S)-2-{[3-({[3-chloro-2′-fluoro-5′-(methyloxy)-4-biphenylyl]sulfonyl}amino)-4-(methyloxy)phenyl]amino}-1-methyl-2-oxoethyl)methylcarbamate (D42)

The title compound (D42) was prepared from the product of Description 38 (D38) and 2-fluoro-5-(methoxy)phenyl]boronic acid using a similar method to that described for Description 39. MS (ES+) m/e 606 [M+H]⁺.

### Description 43

### N-[5-amino-2-(methyloxy)phenyl]-2-chloro-4-(5-methyl-2-furanyl)benzenesulfonamide (D43)

A mixture of N-[5-amino-2-(methyloxy)phenyl]-4-bromo-2-chlorobenzenesulfonamide (391 mg, 1 mmol), 4,4,5,5-tetramethyl-2-(5-methyl-2-furanyl)-1,3,2-dioxaborolane (420 mg, 2 mmol), sodium carbonate (424 mg, 4 mmol) and dichlorobis(triphenylphosphine)palladium (II) (35 mg, 5 mol %) in 1,2-dimethoxyethane (3 mL) and water (1 mL) was microwave heated at 120° C. for 20 minutes. The mixture was diluted with ethyl acetate and washed with water. The organic phase was dried and evaporated. Purification by column chromatography (silica gel) eluting with 25-40% ethyl acetate in hexane gave the title compound (D43). MS (ES+) m/e 359, 361 [M+H]⁺.

### Description 44

### 1,1-Dimethylethyl ((1R)-2-{[3-({[2-chloro-4-(5-methyl-2-furanyl)phenyl]sulfonyl}amino)-4-(methyloxy)phenyl]amino}-1-methyl-2-oxoethyl)methylcarbamate (D44)

A mixture of N-{[(1,1-dimethylethyl)oxy]carbonyl}-N-methyl-D-alanine (50 mg, 0.25 mmol), 4-ethylmorpholine (57 mg, 0.5 mmol), 1-hydroxybenzotriazole hydrate (36 mg, 0.3 mmol), N—[3-(dimethylamino)propyl]-N′-ethylcarbodiimide hydrochloride (57 mg, 0.3 mmol), and N-[5-amino-2-(methyloxy)phenyl]-2-chloro-4-(5-methyl-2-furanyl)benzenesulfonamide (D43) (300 mg, 0.76 mmol) in N,N-dimethylformamide (4 mL) was stirred at room temperature overnight. The mixture was diluted with saturated sodium hydrogen carbonate solution and extracted with ethyl acetate. The organic phase was washed with water and brine, dried and evaporated. Purification on a SCX-2 cartridge eluting with methanol yielded the title compound (D44). MS (ES) m/e 578, 580 [M+H]⁺.

### Description 45

### N-[2-(Methyloxy)-5-nitrophenyl]acetamide (D45)

A mixture of 2-(methyloxy)-5-nitroaniline (3.0 g, 17.8 mmol), acetic anhydride (2.0 g, 1.9 mL, 19.6 mmol), acetic acid (2 mL), toluene (5 mL) and dichloromethane (40 mL) was stirred at room temperature under argon for 90 hours. The reaction was diluted with dichloromethane and saturated sodium bicarbonate solution, and the product extracted into dichloromethane (×2). The combined organic extracts were washed with brine and dried over magnesium sulfate. The solvent was evaporated in vacuo to give the title product (D45). MS (ES+) m/e 211 [M+H]⁺.

### Description 46

### N-[5-Amino-2-(methyloxy)phenyl]acetamide (D46)

A solution of N-[2-(methyloxy)-5-nitrophenyl]acetamide (D45) (1.0 g, 4.8 mmol) in methanol (105 mL) and ethyl acetate (10 mL) was hydrogenated (H-cube, 10% palladium on carbon) at 25° C. and standard pressure. The resulting solution was evaporated in vacuo to afford the title product (D46). MS (ES+) m/e 181 [M+H]⁺.

### Description 47

### N-[5-{[(4-Bromo-3-fluorophenyl)sulfonyl]amino}-2-(methyloxy)phenyl]acetamide (D47)

A mixture of N-[5-amino-2-(methyloxy)phenyl]acetamide (D46) (890 mg, 4.9 mmol), 4-bromo-3-fluorobenzenesulfonyl chloride (1.6 g, 0.88 mL, 5.9 mmol), pyridine (7 mL) and dichloromethane (7 mL) was stirred at room temperature under argon overnight. The reaction was diluted with methanol and the solvent evaporated in vacuo. Dichloromethane and sodium hydrogen carbonate solution were added, and the product extracted into dichloromethane (×2). The combined organic extracts were washed with brine and dried over magnesium sulfate. The solvent was evaporated in vacuo to afford the title product (D47). MS (ES+) m/e 417, 419 [M+H]⁺.

### Description 48

### N-[5-({[3-Fluoro-4-(5-methyl-2-furanyl)phenyl]sulfonyl}amino)-2-(methyloxy)phenyl]acetamide (D48)

A mixture of N-[5-{[(4-bromo-3-fluorophenyl)sulfonyl]amino}-2-(methyloxy)phenyl]acetamide (D47) (600 mg, 1.4 mmol), 4,4,5,5-tetramethyl-2-(5-methyl-2-furanyl)-1,3,2-dioxaborolane (598 mg, 2.9 mmol), sodium carbonate (1.1 g, 10.1 mmol), bis(triphenylphosphine)palladium(II) chloride (105 mg, 0.14 mmol), 1,2-dimethoxyethane (15 mL) and water (10 mL) was heated at reflux under argon overnight. The reaction was cooled to room temperature and then filtered through a pad of celite, washing with ethyl acetate and water. The product was extracted into ethyl acetate (×3), and the combined organic extracts washed with brine and dried over magnesium sulfate. The solvent was evaporated in vacuo to give the crude product, which was purified further by column chromatography (silica gel), eluting with 0-75% ethyl acetate in hexane to afford the title compound (D48). MS (ES+) m/e 419 [M+H]⁺.

### Description 49

### N-[3-Amino-4-(methyloxy)phenyl]-3-fluoro-4-(5-methyl-2-furanyl)benzenesulfonamide (D49)

To N-[5-({[3-fluoro-4-(5-methyl-2-furanyl)phenyl]sulfonyl}amino)-2-(methyloxy)phenyl]acetamide (D48) (300 mg, 0.72 mmol) in ethanol (4 mL) was added 10% sodium hydroxide solution (4 mL), and the reaction stirred at 50° C. under argon overnight. A further portion of 50% sodium hydroxide solution (10 mL) was added and the reaction left at 50° C. overnight. The reaction was cooled to room temperature and the solvent evaporated in vacuo. Ethyl acetate and water were added, and the product extracted into ethyl acetate (×3). The combined organic extracts were washed with brine and dried over magnesium sulfate. The solvent was evaporated in vacuo to afford the title product (D49). MS (ES+) m/e 377 [M+H]⁺.

### Description 50

### 1,1-Dimethylethyl (2-{[5-({[3-fluoro-4-(5-methyl-2-furanyl)phenyl]sulfonyl}amino)-2-(methyloxy)phenyl]amino}-1,1-dimethyl-2-oxoethyl)carbamate (D50)

A mixture of N-[3-amino-4-(methyloxy)phenyl]-3-fluoro-4-(5-methyl-2-furanyl)benzenesulfonamide (D49) (120 mg, 0.32 mmol), N-{[(1,1-dimethylethyl)oxy]carbonyl}-2-methylalanine (518 mg, 2.6 mmol), O-(benzotriazol-1-yl)-N,N—N′,N′-tetramethyluronium hexafluorophosphate (HBTU, 967 mg, 2.6 mmol) and N,N-diisopropylethylamine (330 mg, 0.43 mL, 2.6 mmol) and dichloromethane (3 mL) were stirred at room temperature under argon for 24 hours. Further portions of N-{[(1,1-dimethylethyl)oxy]carbonyl}-2-methylalanine (4 eq.), HBTU (4 eq.), and N,N-diisopropylethylamine (4 eq.) were added and the reaction stirred at room temperature under argon for a further 24 hours and then left to stand for 10 days. The reaction was diluted with dichloromethane and sodium hydrogen carbonate solution, and the product extracted into dichloromethane (×2). The combined organic extracts were washed with brine and dried over magnesium sulfate. The solvent was evaporated in vacuo to give the crude product, which was purified further by column chromatography (silica gel), eluting with 0-40% ethyl acetate in hexanes to afford title compound (D50). MS (ES−) m/e 560 [M−H]⁻.

### Description 51

### 4-Bromo-3-fluoro-N-(3-nitrophenyl)benzenesulfonamide (D51)

A mixture of 3-nitroaniline (600 mg, 4.3 mmol), 4-bromo-3-fluorobenzenesulfonyl chloride (1.4 g, 0.78 mL, 5.2 mmol), pyridine (7 mL) and dichloromethane (7 mL) was stirred at room temperature under argon for 3 hours. The reaction was diluted with dichloromethane and sodium hydrogen carbonate solution, and the product extracted into dichloromethane (×2). The combined organic extracts were washed with brine and dried over magnesium sulfate. The solvent was evaporated in vacuo and co-evaporated with diethyl ether to afford the title product (D51). MS (ES−) m/e 373, 375 [M−H]⁻.

### Description 52

### 3-Fluoro-4-(5-methyl-2-furanyl)-N-(3-nitrophenyl)benzenesulfonamide (D52)

A mixture 4-bromo-3-fluoro-N-(3-nitrophenyl)benzenesulfonamide (D51) (700 mg, 1.9 mmol), 4,4,5,5-tetramethyl-2-(5-methyl-2-furanyl)-1,3,2-dioxaborolane (776 mg, 3.7 mmol), sodium carbonate (1.4 g, 13.1 mmol), bis(triphenylphosphine)palladium(II) chloride (132 mg, 0.19 mmol), 1,2-dimethoxyethane (15 mL) and water (10 mL) was heated at reflux under argon for 3 hours. The reaction was cooled to room temperature and then filtered through a pad of celite, washing with ethyl acetate and water. The filtrate was extracted into ethyl acetate (×3), and the combined organic extracts were washed with brine and dried over magnesium sulfate. The solvent was evaporated in vacuo to give the crude product. Dichloromethane was added and the resulting solid filtered off. The filtrate was purified further by column chromatography (silica gel), eluting with 0-30% ethyl acetate in hexane. The solvent was evaporated in vacuo and combined with the solid from the filtration, and then triturated with diethyl ether. The resulting solid was collected to give the first batch of title compound (D52), and the diethyl ether evaporated in vacuo and further triturated with dichloromethane to afford a second batch of the title compound (D52). MS (ES−) m/e 375 [M−H]⁻.

### Description 53

### N-(3-Aminophenyl)-3-fluoro-4-(5-methyl-2-furanyl)benzenesulfonamide (D53)

To a suspension of 3-fluoro-4-(5-methyl-2-furanyl)-N-(3-nitrophenyl)benzenesulfonamide (D52) (250 mg, 0.93 mmol) in tetrahydrofuran (10 mL) was added palladium on charcoal (10% paste, 20 mg) and the mixture stirred at room temperature under an atmosphere of hydrogen for 80 hours. A further portion of palladium on charcoal (10% paste, 20 mg) was added, and the reaction left for a further 24 hours at room temperature under an atmosphere of hydrogen. The mixture was filtered through celite, washing with tetrahydrofuran and the filtrate was evaporated in vacuo to give the title compound (D53). MS (ES+) m/e 347 [M+H]⁺.

### Description 54

### 1,1-Dimethylethyl (2-{[3-({[3-fluoro-4-(5-methyl-2-furanyl)phenyl]sulfonyl}amino)phenyl]amino}-1,1-dimethyl-2-oxoethyl)carbamate (D54)

A mixture of N-(3-aminophenyl)-3-fluoro-4-(5-methyl-2-furanyl)benzenesulfonamide (130 mg, 0.38 mmol), N-{[(1,1-dimethylethyl)oxy]carbonyl}-2-methylalanine (305 mg, 1.5 mmol), O-(benzotriazol-1-yl)-N,N-N′,N′-tetramethyluronium hexafluorophosphate (HBTU, 569 mg, 1.5 mmol) and N,N-diisopropylethylamine (295 mg, 0.39 mL, 2.3 mmol) and dichloromethane (3 mL) were stirred at room temperature under argon for 24 hours. The reaction was diluted with dichloromethane and sodium hydrogen carbonate solution, and the product extracted into dichloromethane (×3). The combined organic extracts were washed with brine and dried over magnesium sulfate. The solvent was evaporated in vacuo to give the crude product, which was purified further by column chromatography (silica gel), eluting with 0-50% ethyl acetate in hexane to afford the title compound (D54). MS (ES−) m/e 530 [M−H]⁻.

### Description 55

### 4-Bromo-N-(2-chloro-5-nitrophenyl)-3-fluorobenzenesulfonamide (D55)

A mixture of 2-chloro-5-nitroaniline (2.0 g, 11.6 mmol), 4-bromo-3-fluorobenzenesulfonyl chloride (3.8 g, 2.1 mL, 13.9 mmol), pyridine (15 mL) and dichloromethane (15 mL) was stirred at room temperature under argon for 90 hours. A further portion of 4-bromo-3-fluorobenzenesulfonyl chloride (0.6 eq.) was added and the reaction left for a further 4 hours. Methanol was added and the solvent evaporated in vacuo. The resulting solid was triturated with methanol and the solid filtered off. The filtrate was evaporated in vacuo and triturated with dichloromethane. The solid was filtered off and the filtrate purified by column chromatography (silica gel), eluting with 0-15% ethyl acetate in hexane to afford the title compound (D55). MS (ES−) m/e 407, 409 [M−H]⁻.

### Description 56

### N-(5-Amino-2-chlorophenyl)-4-bromo-3-fluorobenzenesulfonamide (D56)

A mixture of 4-bromo-N-(2-chloro-5-nitrophenyl)-3-fluorobenzenesulfonamide (D55) (850 mg, 2.1 mmol), tin(II) chloride dihydrate (2.3 g, 10.4 mmol), ethyl acetate (10 mL) and ethanol (10 mL) was heated at reflux under argon for 3 hours. The reaction was allowed to cool to room temperature and then quenched by the portionwise addition of sodium hydrogen carbonate solution. The reaction mixture was filtered through a pad of celite washing with ethyl acetate (˜300 mL). From the filtrate, the product was extracted into ethyl acetate (×2) and then the combined organic extracts were washed with brine and dried over magnesium sulfate. The solvent was evaporated in vacuo and the resulting solid triturated with dichloromethane and filtered to afford the title compound (D56). MS (ES+) in/e 380 [M+H]⁺.

### Description 57

### N-(5-Amino-2-chlorophenyl)-3-fluoro-4-(5-methyl-2-furanyl)benzenesulfonamide (D57)

A mixture of N-(5-amino-2-chlorophenyl)-4-bromo-3-fluorobenzenesulfonamide (D56) (230 mg, 0.61 mmol), 4,4,5,5-tetramethyl-2-(5-methyl-2-furanyl)-1,3,2-dioxaborolane (252 mg, 1.2 mmol), sodium carbonate (453 mg, 4.3 mmol), bis(triphenylphosphine)palladium(II) chloride (43 mg, 0.06 mmol), 1,2-dimethoxyethane (10 mL) and water (5 mL) was heated at reflux under argon for 2.5 hours. The reaction was cooled to room temperature and then filtered through a pad of celite, washing with ethyl acetate and water. From the filtrate, the product was extracted into ethyl acetate (×2), and the combined organic extracts were washed with brine and dried over magnesium sulfate. The solvent was evaporated in vacuo to give the crude product, which was purified further by column chromatography (silica gel), eluting with 0-35% ethyl acetate in hexane to afford the title compound (D57). MS (ES+) m/e 381 [M+H]⁺.

### Description 58

### 1,1-Dimethylethyl (2-{[4-chloro-3-({[3-fluoro-4-(5-methyl-2-furanyl)phenyl]sulfonyl}amino)phenyl]amino}-1,1-dimethyl-2-oxoethyl)carbamate (D58)

A mixture of N-(5-amino-2-chlorophenyl)-3-fluoro-4-(5-methyl-2-furanyl)benzenesulfonamide (D57) (150 mg, 0.39 mmol), N-{[(1,1-dimethylethyl)oxy]carbonyl}-2-methylalanine (88 mg, 0.43 mmol), O-(benzotriazol-1-yl)-N,N-CAP-tetramethyluronium hexafluorophosphate (HBTU, 163 mg, 0.43 mmol) and N,N-diisopropylethylamine (101 mg, 0.13 mL, 0.78 mmol) and dichloromethane (10 mL) were stirred at room temperature under argon for 18 hours. Further portions of HBTU (4 eq.) and N-{[(1,1-dimethylethyl)oxy]carbonyl}-2-methylalanine (4 eq.) were added and the reaction left for a further 2 hours. Further portions of HBTU (4 eq.) and N-{[(1,1-dimethylethyl)oxy]carbonyl}-2-methylalanine (4 eq.) and N,N-diisopropylethylamine (4 eq.) were added and the reaction left over the weekend. The reaction was diluted with dichloromethane and saturated sodium hydrogen carbonate solution, and the product extracted into dichloromethane (×3). The combined organic extracts were washed with brine and dried over magnesium sulfate. The solvent was evaporated in vacuo to give the crude product, which was purified further by column chromatography (silica gel), eluting with 0-25% ethyl acetate in hexanes. The resulting solid was triturated with diethyl ether to afford the title compound (D58). MS (ES−) m/e 564 [M−H]⁻.

### Description 59

### 4-Bromo-N-[(4-bromo-3-fluorophenyl)sulfonyl]-3-fluoro-N-(2-fluoro-5-nitrophenyl)benzenesulfonamide (D59)

A mixture of 2-fluoro-5-nitroaniline (1.0 g, 6.4 mmol), 4-bromo-3-fluorobenzenesulfonyl chloride (2.1 g, 1.1 mL, 7.7 mmol), pyridine (8 mL) and dichloromethane (8 mL) was stirred at room temperature under argon for 4 hours. A further portion of 4-bromo-3-fluorobenzenesulfonyl chloride (0.3 eq.) was added and the reaction left for a further 15 hours. The solvent was evaporated in vacuo and then dichloromethane and saturated sodium hydrogen carbonate solution were added. The product was extracted into dichloromethane (×3) and the combined organic extracts were washed with brine and dried over magnesium sulfate. The solvent was evaporated in vacuo to afford the title compound (D59). ¹H NMR (CDCl₃): δ 8.43 (1H, m), 8.05 (1H, q), 7.83 (2H, td), 7.74 (2H, dd), 7.61 (2H, dt), 7.38 (1H, t).

### Description 60

### N-(5-Amino-2-fluorophenyl)-4-bromo-N-[(4-bromo-3-fluorophenyl)sulfonyl]-3-fluorobenzenesulfonamide (D60)

A mixture of 4-bromo-N-[(4-bromo-3-fluorophenyl)sulfonyl]-3-fluoro-N-(2-fluoro-5-nitrophenyl)benzenesulfonamide (D59) (600 mg, 0.95 mmol), tin(II) chloride dihydrate (1.1 g, 4.8 mmol), ethyl acetate (7 mL) and ethanol (7 mL) was heated at reflux under argon for 3 hours. The reaction was allowed to cool to room temperature and then quenched by the portionwise addition of saturated sodium hydrogen carbonate solution. The reaction mixture was filtered through a pad of celite washing with ethyl acetate (˜500 mL). The ethyl acetate layer was separated and dried over magnesium sulfate. The solvent was evaporated in vacuo to afford the title compound (D60). MS (ES+) m/e 601 [M+H]⁺.

### Description 61

### 1,1-Dimethylethyl (2-{[4-fluoro-3-({[3-fluoro-4-(5-methyl-2-furanyl)phenyl]sulfonyl}amino)phenyl]amino}-1,1-dimethyl-2-oxoethyl)carbamate (D61)

A mixture of N-(5-amino-2-fluorophenyl)-4-bromo-N-[(4-bromo-3-fluorophenyl)sulfonyl]-3-fluorobenzenesulfonamide (D60) (540 mg, 0.90 mmol), N-{[(1,1-dimethylethyl)oxy]carbonyl}-2-methylalanine (201 mg, 0.99 mmol), O-(benzotriazol-1-yl)-N,N—N′,N′-tetramethyluronium hexafluorophosphate (HBTU, 375 mg, 0.99 mmol) and N,N-diisopropylethylamine (233 mg, 0.31 ml, 1.8 mmol) and dichloromethane (20 ml) were stirred at room temperature under argon for about 90 hours. A further portion of N-{[(1,1-dimethylethyl)oxy]carbonyl}-2-methylalanine (0.6 eq.) and HBTU (0.6 eq.) were added and the reaction left for a further 24 hours. The solvent was evaporated in vacuo and then dichloromethane and saturated sodium hydrogen carbonate solution were added. The product was extracted into dichloromethane (×2) and the combined organic extracts were washed with brine and dried over magnesium sulfate. The solvent was evaporated in vacuo to give a mixture of 1,1-dimethylethyl{2-[(3-{bis[(4-bromo-3-fluorophenyl)sulfonyl]amino}-4-fluorophenyl)amino]-1,1-dimethyl-2-oxoethyl}carbamate and N-(5-amino-2-fluorophenyl)-4-bromo-N-[(4-bromo-3-fluorophenyl)sulfonyl]-3-fluorobenzenesulfonamide (D60) which was used directly. A suspension of N-{[(1,1-dimethylethyl)oxy]carbonyl}-2-methylalanine (274 mg, 1.35 mmol) and ethyl-3-(3-dimethylaminopropyl)carbodiimide hydrochloride (345 mg, 1.8 mmol) in N,N-dimethylformamide (3 ml) was stirred for 15 minutes at room temperature. The crude mixture of 1,1-dimethylethyl {2-[(3-{bis[(4-bromo-3-fluorophenyl)sulfonyl]amino}-4-fluorophenyl)amino]-1,1-dimethyl-2-oxoethyl}carbamate and N-(5-amino-2-fluorophenyl)-4-bromo-N-[(4-bromo-3-fluorophenyl)sulfonyl]-3-fluorobenzenesulfonamide was then added and the reaction left at room temperature for 4.5 hours. Dichloromethane and saturated sodium hydrogen carbonate solution were added, and the product extracted into dichloromethane (×2). The combined organic extracts were washed with brine and dried over magnesium sulfate. The solvent was evaporated in vacuo and to the residue, 4,4,5,5-tetramethyl-2-(5-methyl-2-furanyl)-1,3,2-dioxaborolane (375 mg, 1.8 mmol), sodium carbonate (670 mg, 6.3 mmol), bis(triphenylphosphine)palladium(11) chloride (63 mg, 0.09 mmol), 1,2-dimethoxyethane (16 ml) and water (8 ml) were added. The reaction was heated at reflux under argon overnight. The reaction was then cooled to room temperature and dichloromethane and water added. The reaction mixture was filtered through a pad of celite, washing with dichloromethane and water. From the filtrate, product was extracted into dichloromethane (×3). The combined organic extracts were washed with brine and dried over magnesium sulfate. The solvent was evaporated in vacuo to give crude product, which was purified further by column chromatography (silica gel), eluting with 0-40% ethyl acetate in hexane. The resulting gum was triturated with diethyl ether (×2) to afford the title compound (D61). MS (ES−) m/e 548 [M−H]⁻.

### Description 62

### 6-Bromo-3-(methyloxy)-2-nitropyridine (D62)

A mixture of 6-bromo-3-hydroxy-2-nitropyridine (9.4 g, 42.8 mmol), and potassium carbonate (6.9 g, 50 mmol) in acetone (100 mL) was treated with iodomethane (7.1 g, 3.11 mL, 50 mmol) and stirred at 40° C. Further portions of iodomethane were added as necessary. After 36 hours the solvent was evaporated and the residue was partitioned between ethyl acetate and water. The organic phase was washed with water, dried and evaporated. Purification by column chromatography (silica gel) eluting with 5-50% ethyl acetate in hexane gave the title compound (D62). MS (ES⁺) m/e 233, 235 [M+H]⁺.

### Description 63

### 1,1-Dimethylethyl (1,1-dimethyl-2-{[5-(methyloxy)-6-nitro-2-pyridinyl]amino}-2-oxoethyl)carbamate (D63)

A mixture of 6-bromo-3-(methyloxy)-2-nitropyridine (D62) (500 mg, 2.15 mmol), N²-{[(1,1-dimethylethyl)oxy]carbonyl}-2-methylalaninamide (560 mg, 2.77 mmol), potassium phosphate (1.2 g, 6.0 mmol), copper (I) iodide (114 mg, 0.6 mmol) and N,N′-dimethylethylenediamine (105 mg, 1.2 mmol) in toluene (25 mL) was refluxed for 24 hours. The mixture was cooled, filtered through celite and the solvent evaporated. The crude product was used directly in the next step.

### Description 64

### 1,1-Dimethylethyl (2-{[6-amino-5-(methyloxy)-2-pyridinyl]amino}-1,1-dimethyl-2-oxoethyl)carbamate (D64)

Tin (II) chloride dihydrate (2.2 g, 10 mmol) was added to a solution of 1,1-dimethyl ethyl (1,1-dimethyl-2-{[5-(methyloxy)-6-nitro-2-pyridinyl]amino}-2-oxoethyl)carbamate (300 mg, 0.85 mmol) in ethanol (10 mL). The mixture was refluxed for 2 hours. The mixture was diluted with water and basified by the addition of potassium carbonate. Ethyl acetate was added and the mixture filtered through celite. The organic phase was separated, dried, and evaporated to give an orange oil which was used without further purification.

### Description 65

### 1,1-Dimethylethyl (2-{[6-{[(4-bromo-3-fluorophenyl)sulfonyl]amino}-5-(methyloxy)-2-pyridinyl]amino}-1,1-dimethyl-2-oxoethyl)carbamate (D65)

4-Bromo-3-fluorobenzenesulfonyl chloride (427 mg, 1.5 mmol) was added to a solution of 1,1-dimethylethyl (2-{[6-amino-5-(methyloxy)-2-pyridinyl]amino}-1,1-dimethyl-2-oxoethyl)carbamate (300 mg, 1 mmol) in pyridine (2 mL) and dichloromethane (2 mL). The reaction mixture was stirred at room temperature for 1 hour. The solvent was evaporated and the residue purified by mass directed auto HPLC to give the title compound (D65). MS (ES+) m/e 561, 563 [M+H]⁺.

### Example 1

### N¹-[3-({[3-Fluoro-4-(5-methyl-2-furanyl)phenyl]sulfonyl}amino)-4-(methyloxy)phenyl]-2-methylalaninamide hydrochloride (E1)

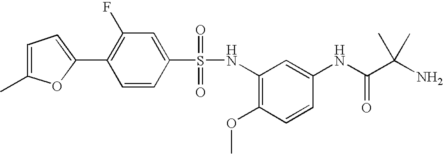

A solution of 1,1-dimethylethyl (2-{[3-({[3-fluoro-4-(5-methyl-2-furanyl)phenyl]sulfonyl}amino)-4-(methyloxy)phenyl]amino}-1,1-dimethyl-2-oxoethyl)carbamate (D4) (400 mg, 0.71 mmol) in dioxane (4 mL) and hydrogen chloride in dioxane (4M solution, 2 mL) was stirred at room temperature for 3 hours. The solvent was evaporated and the residue was co-evaporated with dichloromethane. The residue was dissolved in dichloromethane and the solution was washed with saturated sodium bicarbonate solution, water and brine, dried over anhydrous sodium sulfate and evaporated. The crude product was purified by mass directed auto HPLC. The residue was dissolved in methanol and treated with 1M HCl in ether and the solvent was evaporated. The residue was triturated with ether and the resulting solid was collected and dried to give the title compound (E1). MS (ES+) m/e 462 [M+H]⁺.

### Example 2

### N-[5-(3,3-Dimethyl-2-oxo-1-piperazinyl)-2-(methyloxy)phenyl]-3-fluoro-4-(5-methyl-2-furanyl)benzenesulfonamide hydrochloride (E2)

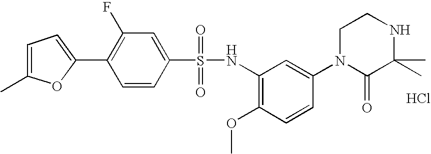

A mixture of 4-bromo-N-[5-(3,3-dimethyl-2-oxo-1-piperazinyl)-2-(methyloxy)phenyl]-3-fluorobenzenesulfonamide (D8) (137 mg, 0.28 mmol) in 1,2-dimethoxyethane (2 mL), (5-methyl-2-furanyl)boronic acid (77 mg, 0.56 mmol), aqueous sodium carbonate solution (1M, 1.4 mL, 1.4 mmol) and bis(triphenylphosphine)palladium(II) chloride (10 mg, 0.014 mmol, 5 mol %) was heated at 120° C. for 20 minutes in a microwave reactor. The crude reaction mixture was applied to an SCX ion exchange cartridge (Varian bond-elute) and washed with methanol and 2M ammonia in methanol. The combined basic fractions were evaporated and the residue purified by column chromatography on silica gel eluting with dichloromethane/2M ammonia in methanol: (20:1 to 10:1). The pure free base from chromatography was dissolved in dichloromethane (2 mL) and treated with 1M HCl in ether (one equivalent). The solvent was evaporated, the residue was triturated with ether and the resulting solid was collected and dried to give the title compound (E2). MS (ES+) m/e 488 [M+H]⁺.

### Examples 3-5

Examples 3-5 (E3-E5) were prepared using a similar method to that described for Description 4 (D4) followed by Example 1 (E1) substituting N-{[(1,1-dimethylethyl)oxy]carbonyl}-2-methylalanine for the appropriate N-protected amino acid indicated in the table:

| | N-Protected Amino | MS |
|---|---|---|
| Example | Acid | [M + H]⁺ |
| 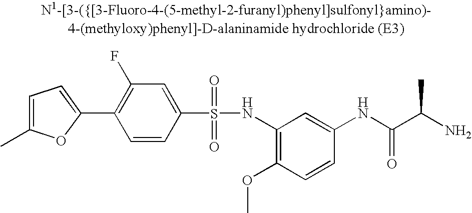 | N-{[(1,1- Dimethylethyl)oxy] carbonyl}-D-alanine | 448 |
| 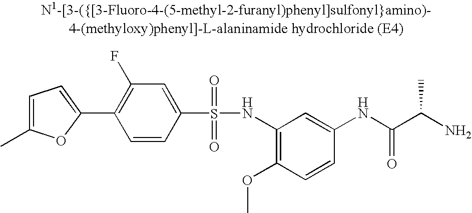 | N-{[(1,1- Dimethylethyl)oxy] carbonyl}-L-alanine | 448 |
| 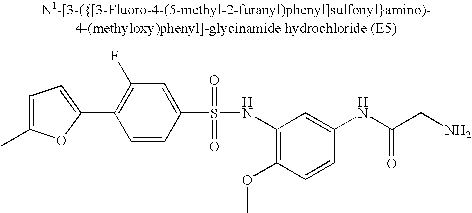 | N-{[(1,1- Dimethylethyl)oxy] carbonyl}-glycine | 434 |

### Example 6

### N¹-[3-({[3-Fluoro-4-(5-methyl-2-furanyl)phenyl]sulfonyl}amino)-4-(methyloxy)phenyl]-N¹,2-dimethylalaninamide hydrochloride (E6)

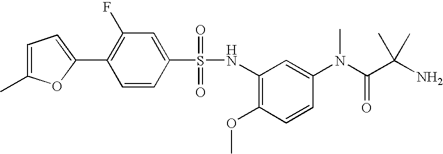

A solution of 1,1-dimethylethyl {2-[[3-({[3-fluoro-4-(5-methyl-2-furanyl)phenyl]sulfonyl}amino)-4-(methyloxy)phenyl](methyl)amino]-1,1-dimethyl-2-oxoethyl}carbamate (D10) (104 mg, 0.18 mmol) in dioxane (4 mL) was treated with hydrogen chloride in dioxane (4M solution, 2 mL) and the mixture was stirred at room temperature for 3 hours. The solvent was evaporated and the residue was co-evaporated with methanol and then diethyl ether. The residue was purified by column chromatography (silica gel) eluting with dichloromethane/2M ammonia in methanol (20:1 to 10:1). The free base was dissolved in dichloromethane and treated with 1M HCl in ether to give the title product (E6). MS (ES+) m/e 476 [M+H]⁺.

### Example 7

### (2S)-2-Amino-N-[3-({[3-fluoro-4-(5-methyl-2-furanyl)phenyl]sulfonyl}amino)-4-(methyloxy)phenyl]butanamide hydrochloride (E7)

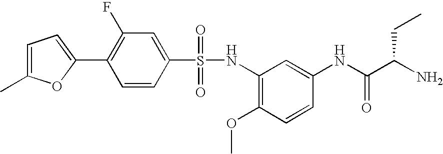

1,1-Dimethylethyl [(1S)-1-({[3-({[3-fluoro-4-(5-methyl-2-furanyl)phenyl]sulfonyl}amino)-4-methyloxy)phenyl]amino}carbonyl)propyl]carbamate (D12) (200 mg, 0.36 mmol) was stirred at room temperature for 1 hour in a solution of hydrogen chloride in dioxane (4M solution, 2 mL). The solvent was evaporated in vacuo and the residue was purified by SCX cartridge followed by mass directed auto HPLC. The residue was dissolved in methanol (1 mL), treated with 1M HCl in ether (0.3 mL) and stirred at room temperature for 10 minutes. The solvent was evaporated in vacuo and the residue triturated with ether to give the title compound (E7). MS (ES+) m/e 462 [M+H]⁺.

### Example 8

### N¹-[3-({[3-Fluoro-4-(5-methyl-2-furanyl)phenyl]sulfonyl}amino)-4-(methyloxy)phenyl]-L-serinamide hydrochloride (E8)

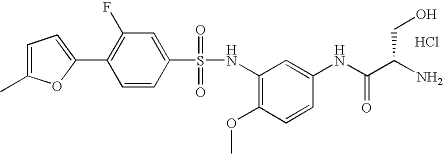

1,1-Dimethylethyl [(1S)-2-{[3-({[3-fluoro-4-(5-methyl-2-furanyl)phenyl]sulfonyl}amino)-4-(methyloxy)phenyl]amino}-1-(hydroxymethyl)-2-oxoethyl]carbamate (D13) (0.54 mmol, 0.304 g) was dissolved in hydrogen chloride in dioxane (4M solution, 8 mL) and stirred under argon for 1 hour. The solvent was removed in vacuo and the residue was dissolved in methanol and loaded onto a SCX cartridge. It was washed with methanol (80 mL) and eluted with 2M ammonia in methanol (80 mL), the basic fractions were combined and concentrated. The residue was dissolved in a minimum volume of dichloromethane, treated with 1M HCl in diethyl ether and the solvent removed in vacuo. It was then further purified by mass directed auto HPLC. The residue was dissolved in dichloromethane and was treated with excess 1M HCl in diethyl ether. The solvent was removed via a stream of compressed air to give the title compound (E8). MS (ES+) m/e 464 [M+H]⁺.

### Example 9

### N¹-[3-({[3-Fluoro-4-(5-methyl-2-furanyl)phenyl]sulfonyl}amino)-4-(methyloxy)phenyl]-2-methylserinamide hydrochloride (E9)

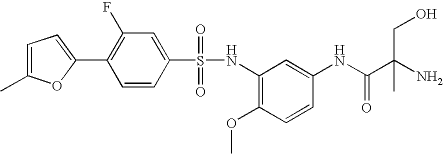

The 1,1-dimethylethyl [2-{[3-({[3-fluoro-4-(5-methyl-2-furanyl)phenyl]sulfonyl}amino)-4-(methyloxy)phenyl]amino}-1-(hydroxymethyl)-1-methyl-2-oxoethyl]carbamate (D15) (0.39 mmol, 0.226 g) was treated with a solution of hydrogen chloride in dioxane (4M solution, 5 mL) and was stirred for 1 hour. The reaction mixture was evaporated and purified by mass directed auto HPLC. The residue was redissolved in methanol and treated with excess 1M HCl in diethyl ether, then evaporated to give the title compound (E9). MS (ES+) m/e 478 [M+H]⁺.

### Example 10

### N¹-[3-({[3-Fluoro-4-(5-methyl-2-furanyl)phenyl]sulfonyl}amino)-4-(methyloxy)phenyl]-N²-methyl-L-alaninamide hydrochloride (E10)

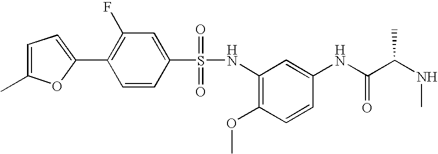

The title compound (E10) was prepared from the product of Description 16 (D16) in a similar method to that described for Example 7. MS (ES+) m/e 462 [M+H]⁺.

### Example 11

### N¹-[3-({[3-Fluoro-4-(5-methyl-2-furanyl)phenyl]sulfonyl}amino)-4-(methyloxy)phenyl]-N²,2-dimethylalaninamide hydrochloride (E11)

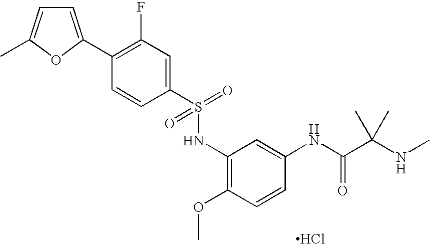

A solution of N-[5-amino-2-(methyloxy)phenyl]-3-fluoro-4-(5-methyl-2-furanyl)benzenesulfonamide (D19) (200 mg, 0.53 mmol) in dichloromethane (4 mL) was treated with N,N-diisopropylethylamine (185 ul, 1.06 mmol), N-{[(1,1-dimethylethyl)oxy]carbonyl}-N,2-dimethylalanine (140 mg, 0.64 mmol) and O-(benzotriazol-1-yl)-N,N—N′,N′-tetramethyluronium hexafluorophosphate (HBTU, 242 mg, 0.64 mmol). The mixture was stirred at room temperature over the weekend. Additional quantities of N-{[(1,1-dimethylethyl)oxy]carbonyl}-N,2-dimethylalanine (140 mg, 0.64 mmol) and O-(benzotriazol-1-yl)-N,N—N′,N′-tetramethyluronium hexafluorophosphate (HBTU, 242 mg, 0.64 mmol) were added. After stirring at room temperature again overnight, the reaction mixture was purified by SCX cartridge eluting with methanol then 2M ammonia in methanol. The basic fractions were then combined and evaporated. The residue was purified by column chromatography (silica gel) eluting with 20-60% ethyl acetate in hexanes then 0-5% 2M ammonia in methanol/dichloromethane. The solid was dissolved in methanol (1 mL), treated with 1M HCl in ether (0.3 mL) and stirred at room temperature for 15 minutes. The solvent was evaporated in vacuo, the solid triturated with ether then further purified by mass directed auto HPLC. The resulting solid was then dissolved in methanol (1 mL), treated with 1M HCl in ether (0.3 mL). The solvent was evaporated in vacuo to give the title product (E11). MS (ES+) m/e 475 [M+H]⁺.

### Example 12

### N¹-[3-({[2-Chloro-4-(5-methyl-2-furanyl)phenyl]sulfonyl}amino)-4-(methyloxy)phenyl]-2-methylalaninamide hydrochloride (E12)

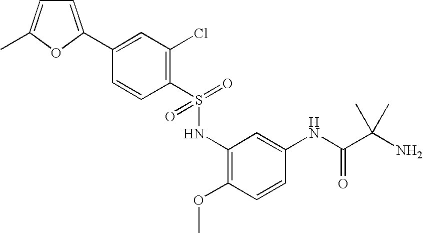

The N¹-[3-{[(4-bromo-2-chlorophenyl)sulfonyl]amino}-4-(methyloxy)phenyl]-2-methylalaninamide (D22) (0.04 g, 0.16 mmol), 4,4,5,5-tetramethyl-2-(5-methyl-2-furanyl)-1,3,2-dioxaborolane (0.025 g, 0.12 mmol), dichlorobis(triphenylphosphine)palladium (II) (0.003 g, 0.004 mmol) and sodium carbonate (0.034 g, 0.32 mmol) in 1,2-dimethoxyethane (2 mL)/water (1 mL), were heated at 120° C. for 20 minutes in the microwave reactor. The 1,2-dimethoxyethane/water was removed in vacuo and the resulting residue was partitioned between diethyl ether and saturated hydrogen carbonate solution. The organics were separated and washed further with brine, dried over sodium sulfate and concentrated in vacuo. The resulting residue was purified via mass directed auto HPLC. The residue was re-evaporated from toluene (×3) and then dissolved in 1:1 methanol/dichloromethane and treated with excess 1M HCl in diethyl ether to give the title compound (E12). MS (ES+) m/e 478 [M+H]⁺.

### Example 13

### 2-Methyl-N¹-[3-({[4-(5-methyl-2-furanyl)phenyl]sulfonyl}amino)-4-(methyloxy)phenyl]alaninamide hydrochloride (E13)

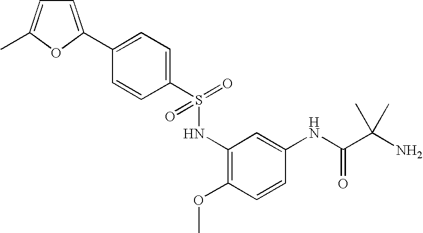

The title compound (E13) was prepared from the product of Description 25 (D25) in a similar method to that described for Example 12. MS (ES+) m/e 444 [M+H]⁺.

### Example 14

### N¹-[3-({[2-Chloro-4-(3-furanyl)phenyl]sulfonyl}amino)-4-(methyloxy)phenyl]-2-methylalaninamide hydrochloride (E14)

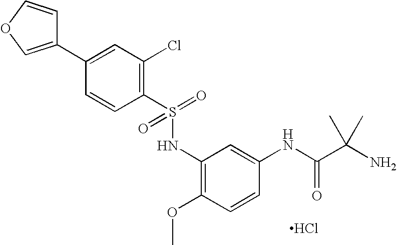

The title compound (E14) was prepared from the product of Description 22 (D22) in a similar method to that described for Example 12 substituting 4,4,5,5-tetramethyl-2-(5-methyl-2-furanyl)-1,3,2-dioxaborolane with 2-(3-furanyl)-4,4,5,5-tetramethyl-1,3,2-dioxaborolane. MS (ES+) m/e 464 [M+H]⁺.

### Example 15

### N¹-[3-({[3-Fluoro-4-(3-furanyl)phenyl]sulfonyl}amino)-4-(methyloxy)phenyl]-2-methylalaninamide hydrochloride (E15)

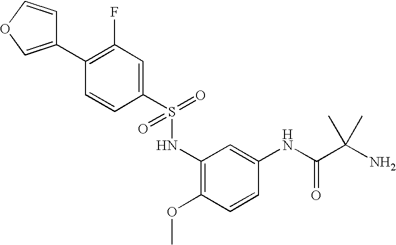

The title compound (E14) was prepared from the product of Description 28 (D28) in a similar method to that described for Example 12 substituting 4,4,5,5-tetramethyl-2-(5-methyl-2-furanyl)-1,3,2-dioxaborolane with 2-(3-furanyl)-4,4,5,5-tetramethyl-1,3,2-dioxaborolane. MS (ES+) m/e 448 [M+H]⁺.

### Example 16

### N¹-[3-({[2-Chloro-4-(5-methyl-2-furanyl)phenyl]sulfonyl}amino)-4-(methyloxy)phenyl]-N²-methyl-L-alaninamide hydrochloride (E16)

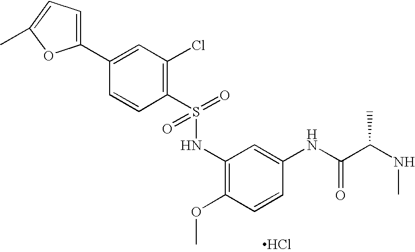

1,1-Dimethyl ethyl ((1S)-2-[3-({[2-chloro-4-(5-methyl-2-furanyl)phenyl]sulfonyl}amino)-4-(methyloxy)phenyl]amino-1-methyl-2-oxoethyl)methylcarbamate (D30) (0.089 g, 0.154 mmol) was treated with hydrogen chloride in dioxane (4M solution, 5 mL) and stirred at 44° C. for 1 hour. The reaction mixture was evaporated to a minimum and was purified by mass directed auto HPLC. The product was redissolved in methanol and treated with excess 1M HCl in diethyl ether, then evaporated and the resultant oil solidified by triturating with diethyl ether/ethyl acetate (3×5 mL) to give the title compound (E16). MS (ES+) m/e 478/480 [M+H]⁺.

### Example 17

### N¹-[3-({[4-(5-Methyl-2-furanyl)phenyl]sulfonyl}amino)-4-(methyloxy)phenyl]-D-alaninamide hydrochloride (E17)

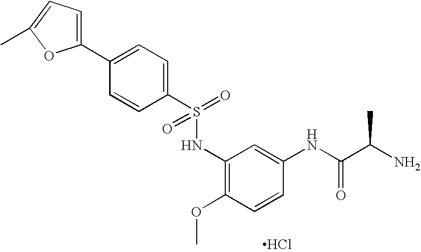

1,1-Dimethylethyl ((1R)-1-methyl-2-{[3-({[4-(5-methyl-2-furanyl)phenyl]sulfonyl}amino)-4-(methyloxy)phenyl]amino}-2-oxoethyl)carbamate (D32) (105 mg) was treated with hydrogen chloride in dioxane (4M solution, 3 mL) and the resulting solution was diluted with further dioxane (5 mL). The reaction mixture was stirred at room temperature for 2 h and then more hydrogen chloride in dioxane (4M solution, 1 mL) was added. The reaction mixture was stirred overnight at room temperature and then evaporated to dryness to afford the title compound (E17). MS (ES+) 430 [M+H]⁺.

### Example 18

### N¹-[3-({[2-Chloro-4-(5-methyl-2-furanyl)phenyl]sulfonyl}amino)-4-(methyloxy)phenyl]-D-alaninamide hydrochloride (E18)

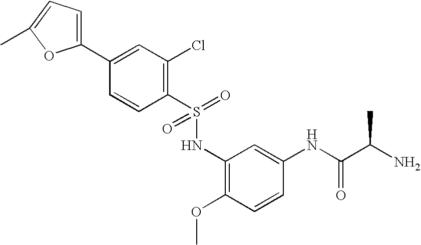

1,1-Dimethyl ethyl ((1R)-2-{[3-({[2-chloro-4-(5-methyl-2-furanyl)phenyl]sulfonyl}amino)-4-(methyloxy)phenyl]amino}-1-methyl-2-oxoethyl)carbamate (D34) (52 mg) was treated with hydrogen chloride in dioxane (4M solution, 2 mL) and the resulting solution was diluted with further dioxane (5 mL). The reaction mixture was stirred at room temperature for 2 hours and then more hydrogen chloride in dioxane (4M solution, 1 mL) was added. The reaction mixture was stirred overnight at room temperature and then evaporated to dryness to afford the crude title compound. This was purified by mass directed auto HPLC and the pure fractions were evaporated to dryness. The residue was redissolved in methanol and treated with excess 1M HCl in diethyl ether. The solvent was removed in vacuo to afford the title compound (E18). MS (ES+) 464/466 [M+H]⁺.

### Example 19

### N¹-[3-({[2-Chloro-4-(5-methyl-2-furanyl)phenyl]sulfonyl}amino)-4-(methyloxy)phenyl]-L-alaninamide hydrochloride (E19)

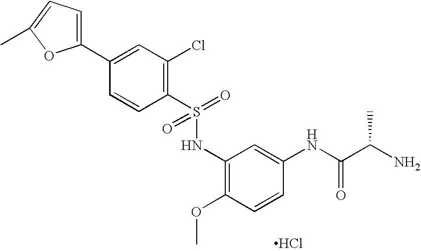

1,1-Dimethylethyl ((1S)-2-{[3-({[2-chloro-4-(5-methyl-2-furanyl)phenyl]sulfonyl}amino)-4-(methyloxy)phenyl]amino}-1-methyl-2-oxoethyl)carbamate (D36) (95 mg) was treated with hydrogen chloride in dioxane (4M solution, 3 mL) and the resulting solution was diluted with further dioxane (5 mL). The reaction mixture was stirred overnight at room temperature and then evaporated to dryness to afford the title compound (E19). MS (ES+) 464/466 [M+H]⁺.

### Example 20

### N¹-[3-({[2-Chloro-4-(4-methyl-2-thienyl)phenyl]sulfonyl}amino)-4-(methyloxy)phenyl]-N²-methyl-L-alaninamide hydrochloride (E20)

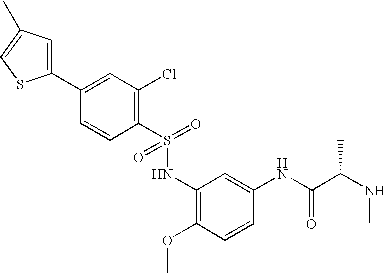

1,1-Dimethylethyl ((1S)-2-{[3-({[2-chloro-4-(4-methyl-2-thienyl)phenyl]sulfonyl}amino)-4-(methyloxy)phenyl]amino}-1-methyl-2-oxoethyl)methylcarbamate (D37) (0.082 g, 0.138 mmol) was dissolved in hydrogen chloride in dioxane (4M solution, 5 mL) and stirred at 44° C. for 1 hour. The reaction mixture was evaporated to a minimum and purified by mass directed auto HPLC. The isolated product was evaporated to a minimum, redissolved in methanol and treated with excess 1M HCl in diethyl ether, then evaporated and the resultant oil solidified by triturating with diethyl ether/ethyl acetate (3×5 mL) to give the title compound (E20). MS (ES+) m/e 494/496 [M+H]⁺.

### Example 21

### N¹-[3-({[3-Chloro-3′-(methyloxy)-4-biphenylyl]sulfonyl}amino)-4-(methyloxy)phenyl]-N²-methyl-L-alaninamide hydrochloride (E21)

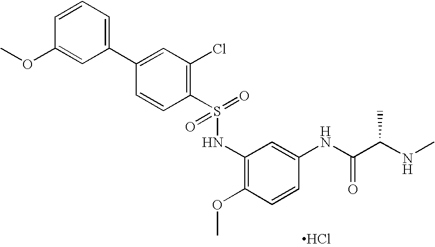

1,1-Dimethylethyl ((1S)-2-{[3-({[3-chloro-3′-(methyloxy)-4-biphenylyl]sulfonyl}amino)-4-(methyloxy)phenyl]amino}-1-methyl-2-oxoethyl)methylcarbamate (D39) (0.22 mmol, 0.134 g) was dissolved in hydrogen chloride in dioxane (4M solution, 3 mL) and stirred for 1 hour. The solvent was removed in vacuo to give the title compound. MS (ES+) m/e 504 [M+H]⁺.

### Example 22

### N¹-[3-({[2′-Fluoro-5′-(methyloxy)-4-biphenylyl]sulfonyl}amino)-4-(methyloxy)phenyl]-N²-methyl-L-alaninamide hydrochloride (E22)

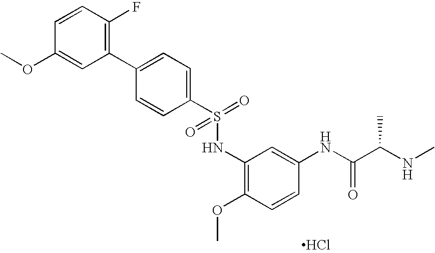

The title compound (E22) was prepared from the product of Description 41 (D41) in a similar method to that described for Example 21. MS (ES+) m/e 488 [M+H]⁺.

### Example 23

### N¹-[3-({[3-Chloro-2′-fluoro-5′-(methyloxy)-4-biphenylyl]sulfonyl}amino)-4-(methyloxy)phenyl]-N²-methyl-L-alaninamide hydrochloride (E23)

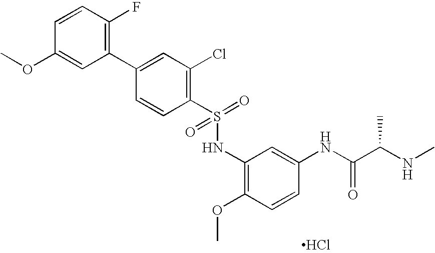

The title compound (E23) was prepared from the product of Description 42 (D42) in a similar method to that described for Example 21. MS (ES+) m/e 522 [M+H]⁺.

### Example 24

### N¹-[3-({[2-Chloro-4-(5-methyl-2-furanyl)phenyl]sulfonyl}amino)-4-(methyloxy)phenyl]-N²-methyl-D-alaninamide hydrochloride (E24)

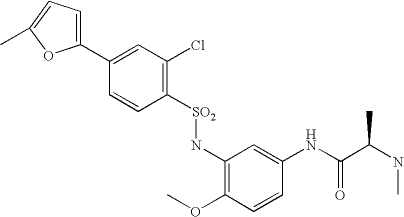

A solution of 1,1-dimethylethyl ((1R)-2-{[3-({[2-chloro-4-(5-methyl-2-furanyl)phenyl]sulfonyl}amino)-4-(methyloxy)phenyl]amino}-1-methyl-2-oxoethyl)methylcarbamate (D44) (90 mg, 0.16 mmol) in dioxan (2 mL) was treated with hydrogen chloride in dioxane (4M solution, 1 mL) and stirred at room temperature for 2 hours. Diethyl ether was added and the precipitate filtered off. Purification by mass-directed auto HPLC and conversion to the hydrochloride salt by treatment with 1M HCl in diethyl ether gave the title compound (E24). MS (ES) m/e 478, 480 [M+H]⁺.

### Example 25

### N¹-[5-({[3-Fluoro-4-(5-methyl-2-furanyl)phenyl]sulfonyl}amino)-2-(methyloxy)phenyl]-2-methylalaninamide hydrochloride (E25)

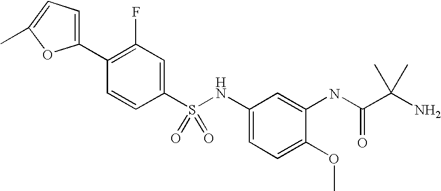

To 1,1-dimethylethyl (2-{[5-({[3-fluoro-4-(5-methyl-2-furanyl)phenyl]sulfonyl}amino)-2-(methyloxy)phenyl]amino}-1,1-dimethyl-2-oxoethyl)carbamate (D50) (130 mg, 0.23 mmol) in dioxane (2 mL) was added hydrogen chloride in dioxane (4M solution, 2.5 mL), and the reaction stirred at room temperature under argon for 3 hours. The solvent was evaporated in vacuo. The residue was dissolved in methanol and purified by SCX, eluting with methanol and then with ammonia in methanol solution (2M). The basic fractions were combined and solvent evaporated in vacuo. The crude product was purified further by mass directed auto HPLC. The residue was dissolved in methanol (1 mL) and dichloromethane (1 mL), and treated with 1M HCl in ether (0.5 mL). The solvent was evaporated to afford the title compound (E25). MS (ES+) m/e 462 [M+H]⁺.

### Example 26

### N¹-[3-({[3-Fluoro-4-(5-methyl-2-furanyl)phenyl]sulfonyl}amino)phenyl]-2-methylalaninamide hydrochloride (E26)

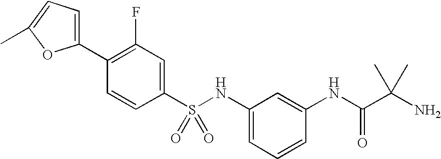

To 1,1-dimethylethyl (2-{[3-({[3-fluoro-4-(5-methyl-2-furanyl)phenyl]sulfonyl}amino)phenyl]amino}-1,1-dimethyl-2-oxoethyl)carbamate (D54) (170 mg, 0.32 mmol) in dioxane (2 mL) was added hydrogen chloride in dioxane (4M solution, 2.5 mL), and the reaction stirred at room temperature under argon for 4 hours. The solvent was evaporated in vacuo. The residue was dissolved in methanol and purified by SCX, eluting with methanol and then with ammonia in methanol solution (2M). The basic fractions were combined and the solvent evaporated in vacuo. The crude product was purified further by mass directed auto HPLC. The residue was dissolved in methanol (0.5 mL) and dichloromethane (1 mL), and treated with 1M HCl in ether (0.5 mL). The solvent was evaporated to afford the title compound (E26). MS (ES+) m/e 432 [M+H]⁺.

### Example 27

### N¹-[4-Chloro-3-({[3-fluoro-4-(5-methyl-2-furanyl)phenyl]sulfonyl}amino)phenyl]-2-methylalaninamide hydrochloride (E27)

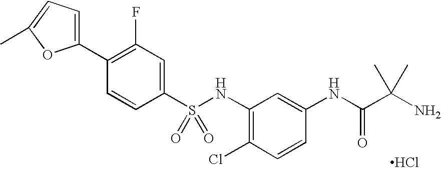

To 1,1-dimethylethyl (2-{[4-chloro-3-({[3-fluoro-4-(5-methyl-2-furanyl)phenyl]sulfonyl}amino)phenyl]amino}-1,1-dimethyl-2-oxoethyl)carbamate (D58) (65 mg, 0.11 mmol) in dioxane (2 mL) was added hydrogen chloride in dioxane (4M solution, 1.5 mL), and the reaction stirred at room temperature under argon for 2 hours. The solvent was evaporated in vacuo, azeotroped with dichloromethane (×2) and the residue triturated with diethyl ether to afford the title compound (E27). MS (ES+) m/e 466 [M+H]⁺.

### Example 28

### N¹-[4-Fluoro-3-({[3-fluoro-4-(5-methyl-2-furanyl)phenyl]sulfonyl}amino)phenyl]-2-methylalaninamide hydrochloride (E28)

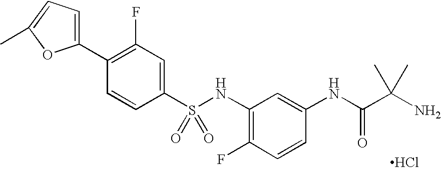

To 1,1-dimethylethyl (2-{[4-fluoro-3-({[3-fluoro-4-(5-methyl-2-furanyl)phenyl]sulfonyl}amino)phenyl]amino}-1,1-dimethyl-2-oxoethyl)carbamate (D61) (65 mg, 0.12 mmol) in dioxane (2 mL) was added hydrogen chloride in dioxane (4M solution, 1.5 mL), and the reaction stirred at room temperature under argon for 1.5 hours. The solvent was evaporated in vacuo, co-evaporated with dichloromethane (×4) and then triturated with diethyl ether to afford the title compound (E28). MS (ES+) m/e 450 [M+H]⁺.

### Example 29

### N¹-[6-({[3-Fluoro-4-(5-methyl-2-furanyl)phenyl]sulfonyl}amino)-5-(methyloxy)-2-pyridinyl]-2-methylalaninamide hydrochloride (E29)

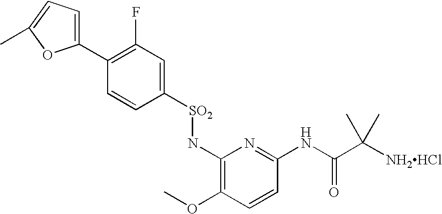

A mixture of 1,1-dimethylethyl (2-{[6-{[(4-bromo-3-fluorophenyl)sulfonyl]amino}-5-(methyloxy)-2-pyridinyl]amino}-1,1-dimethyl-2-oxoethyl)carbamate (D65) (30 mg, 0.05 mmol), 4,4,5,5-tetramethyl-2-(5-methyl-2-furanyl)-1,3,2-dioxaborolane (22 mg, 0.1 mmol), sodium carbonate (21 mg, 0.2 mmol) and dichlorobis(triphenylphosphine)palladium (II) (2 mg, 5 mol %) in 1,2-dimethoxyethane (2 mL) and water (0.5 mL) was microwave heated at 120° C. for 20 minutes. The mixture was diluted with diethyl ether and washed with water. The organic phase was dried and evaporated. The residue was dissolved in dioxan (1 mL) and hydrogen chloride in dioxan (4M solution, 0.5 mL) was added. The mixture was stirred at room temperature for 2 hours. The solvent was evaporated and triturated with diethyl ether to give the title compound (E29). MS (ES⁺) m/e 463 [M+H]⁺.

### Assay Procedures

### Cloning of the Ghrelin Receptor GHS-R

Human GHS-R was cloned from human hypothalamus cDNA and TOPO Ta cloned into pCR2.1. The sequence was confirmed and transferred into pCDN for expression analysis. The sequence was confirmed again and the plasmid was electroporated into CHO cells. The clones were screened by FLIPR (Fluorometric Imaging Plate Reader).

### Generation of the GHS-R Bacmam Virus and Viral Titre Determination

### Virus Generation

The open reading frame of GHS-R was transferred from pCDN into pFastBacmam vector. This vector was used to generate recombinant baculoviruses in which the insect cell-specific polyhedrin promoter has been replaced with a mammalian cell-active promoter, in this case CMV. This was then used with the Bac to Bac expression system (Invitrogen). Briefly the vector was transformed into DH10 bac E. coli and the bacmid isolated from the transformed cells. The bacmid was then transfected into Sf9 insect cells grown in ExCell 420 (JRH) medium in 6-well dishes for the production of recombinant baculovirus particles.

The supernatant from these cells was harvested containing the recombinant GHS-R bacmam virus. This P0 viral stock was then used to infect 200 mLs of 1×10⁻⁶ cells/mL Sf9 cells in ExCell 420 medium to further amplify the virus and provide a P1 stock.

This P1 viral stock was then used to amplify a P2 viral stock of 10×1 litre erlenmeyer shake flasks again harvesting the supernatant from the cells. This was then used to transduce mammalian cells for assay.

The open reading frame of rat Gαo G-protein was cloned by PCR from rat brain cDNA into pcDNA3 vector. This was then transferred into the pFast Bacmam vector and recombinant baculovirus particles generated as above.

### Viral Titre Determination

Viral titres were determined at all stages of the virus scale up with a plaque ELISA method using a gp64 envelope protein monoclonal antibody.

SF9 cells were plated out into a 96 well plate and a dilution range of virus was added to the cells for 1 hour. The virus was removed and a 1% methylcellulose and media mix was added to the cells and incubated for 48 hrs. The cells were then fixed in a formaldehyde and acetone mix for 8 minutes. The cells were then washed with a phosphate buffered saline solution (PBS) and normal goat serum added for 25 mins. This was then removed and a gp64 monoclonal antibody added for 25 mins. The wells were then washed with PBS and a goat anti-mouse/HRP conjugated antibody added for 25 mins The wells were again washed with PBS and True Blue peroxidase substrate solution (Kirkegaard & Perry Laboratories) added and incubated for 60 mins.

Individual wells were counted for blue foci and taking into account the dilution factor, the plaque forming units/mL of the virus was determined.

### 1. GHS-R GTPγS Functional Agonist Assay

### Generation of Cells Transiently Expressing the Ghrelin Receptor GHS-R

HEK293T cells (HEK293 cells stably expressing the SV40 large T-antigen) were maintained in DMEM containing 10% (v/v) newborn calf serum and 2 mM glutamine. Cells were seeded in 60 mm culture dishes and grown to 60-80% confluency (18-24 hrs) prior to transfection with pcDNA3 containing the relevant DNA species using Lipofectamine reagent. For transfection, 3 μg of DNA was mixed with 10 μl of Lipofectamine in 0.2 mL of Opti-MEM (Life Technologies Inc.) and was incubated at room temperature for 30 min prior to the addition of 1.6 mL of Opti-MEM. For cotransfection experiments, 1.5 μg of each cDNA species was used. Cells were exposed to the Lipofectamine/DNA mixture for 5 hrs and 2 mL of 10% (v/v) newborn calf serum in DMEM was then added. Cells were harvested 48 hrs after transfection.

### Generation of Cells Transiently Expressing the Ghrelin Receptor GHS-R and Rat Gαo G-protein.

HEK293F cells maintained in Freestyle media (Invitrogen) were co-transduced with both GHS-R and rat Gαo G-protein by adding 300 mLs of GHS-R virus (1×10⁸ pfu/mL) and 30 mLs of Gαo G-protein (4×10⁸ pfu/mL) to 3×10⁸ HEKF cells in 1 litre of freestyle media. 24 hours post transduction 2 mM sodium butyrate was added to enhance expression. 24 hours post sodium butyrate addition. The cells were harvested by membrane preparation.

Membrane Preparation from Cultured Cells

All steps of the protocol are carried out at 4° C. and with pre-cooled reagents. The cell pellet was resuspended in 10 volumes of buffer A2 containing 50 mM N-2-hydroxyethylpiperazine-N′-2-ethanesulfonic acid (HEPES) (pH 7.40) supplemented with 10e-4M leupeptin (acetyl-leucyl-leucyl-arginal; Sigma L2884), 25 μg/mL bacitracin (Sigma B0125), 1 mM ethylenediamine tetra-acetic acid (EDTA), 1 mM phenylmethylsulfonyl fluoride (PMSF) and 2×10e-6M pepstain A (Sigma). The cells were then homogenised by 2×15 sec bursts in a 1 litre glass Waring blender, followed by centrifugation at 500 g for 20 mins. The supernatant was then spun at 48,000 g for 30 mins. The pellet was resuspended in 4 volumes of buffer A2 by vortexing for 5 secs, followed by homogenisation in a Dounce homogeniser (10-15 strokes). At this point the preparation was aliquoted into polypropylene tubes and stored at −70° C.

Compounds of the invention were tested for in vitro biological activity in accordance with the following GTPγS assay:

### GHS-R GTPγS Functional Agonist Assay Protocol

For each compound being assayed, in an Opti clear bottom 96 well plate, is added:—

(a) 5 μl of test compound diluted to required concentration in 100% DMSO and added to 15 μl assay buffer (20 mM N²-Hydroxyethylpiperazine-N′-2-ethanesulfonic acid (HEPES)+100 mM NaCl+10 mM MgCl₂, pH adjusted to 7.4 with NaOH);
(b) 20 μl guanosine 5′ [γ35-S]thiotriphosphate, triethylamine salt (Amersham; radioactivity concentration=37 kBq/μl or 1 mCi/mL; Specific Activity 1160 Ci/mmol) diluted to 1.9 nM in assay buffer to give 0.38 nM final.
(c) Membrane (prepared in accordance with the methodology described above) were diluted in assay buffer to give a final concentration which contains 5 μg protein per well in 60 μl. 40 μM final concentration of guanosine 5′ diphosphate (GDP) (Sigma; diluted in assay buffer) was added and left to incubate for 10 minutes before addition to the assay

The assay is started by the mixing of components from a, b and c and allowed was to incubated at room temperature for 30 mins.

(d) Wheat germ agglutinin-polyvinyltoluene (WGA-PVT) scintillation proximity assay (SPA) beads were diluted in assay buffer to a concentration of 20 mgs/mL.

25 μl of bead was then added to the reaction mix and the assay was incubated for another 30 mins at room temperature with shaking. This was followed by centrifugation for 5 mins at 1500 rpm. The plate was read between 3 and 6 hours after completion of centrifuge run in a Wallac Microbeta counter on a 1 min normalised tritium count protocol. Data was analysed using a 4-parameter logistic equation. Basal activity used as minimum.

The Examples have activities of <1 μM in the GHS-R GTPγS functional agonist assays.

### 2. GHSR Agonist BACMAM FLIPR Assay

### Generation of U2Os Cells Transiently Expressing the Ghrelin Receptor GHS-R

24 hours prior to assay U2OS cells at confluence 100% are harvested and spun down. The supernatant is removed and the cells resuspended in media (DMEM+10% FBS+1% L-Glutamine). A cell count is performed using the Cedex instrumentation, and the concentration of cells is adjusted using media to give 20K cells per mL (10K cells/50 μl).

Human GHSR BACMAM virus is added to the cell suspension at an appropriate % volume (calculated for individual batches of BACMAM virus as viral titres vary). The transduced cell suspension is dispensed into FLIPR 384-well clear bottom plates, 50 ul per well. Cell plates are incubated at 37° C. overnight.

### Compound Preparation

Master compound plates are prepared in 100% DMSO. 3 mM is the top concentration (giving 10 μM final concentration) and they are serially diluted 1 in 4. 1 ul from the master plate is transferred to a daughter plate, to which is added 50 μl of compound dilution buffer (Tyrodes+1 mg/mL BSA+1.5 mM CaCl₂). This plate is used for the assay.

Compounds of the invention were tested for in vitro biological activity in accordance with the following FLIPR assay:

### GHSR Agonist BACMAM FLIPR Assay Protocol

Media is aspirated from cell plates using a cell washer (leaving 10 ul of media). Cells are immediately loaded with loading buffer (Tyrodes (Elga water+145 mM NaCl+5 mM KCl+20 mM HEPES+10 mM glucose+1 mM MgCl₂)+1.5 mM CaCl₂+0.714 mg/mL Probenicid (predissolved in 1 M NaOH)+0.5 mM brilliant black+2.5 uM Fluo 4 dye, and incubated at 37.5° C. for 1 hour. 10 μl from compound plates is then added immediately to cell plates using a FLIPR 3 calcium imaging instrument. Fluorescence measurements are taken.

The Examples have an EC₅₀ values of <1 μM in the GHSR Agonist BACMAM FLIPR Assay.

What is claimed is:

1. A compound of formula (I) or a pharmaceutically acceptable salt thereof: 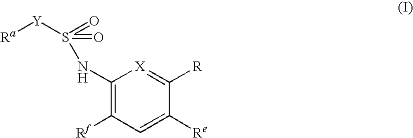
in which R^{a} is aryl or heteroaryl;
Y is a single bond, CH₂, CH₂CH₂, or CH═CH;
X is CH or N;
R^{e} is hydrogen, C₁₋₆alkyl, C₃₋₆cycloalkyl, COC₁₋₆alkyl, C₁₋₆alkoxy, halogen, hydroxyl, trifluoromethyl, trifluoromethoxy or cyano;
R^{f} is hydrogen, C₁₋₆alkyl, C₃₋₆cycloalkyl, COC₁₋₆alkyl, C₁₋₆alkoxy, C₁₋₆alkoxyC₁₋₆alkyl, halogen, hydroxyl, trifluoromethyl, or cyano;
R is a group of formula (A): 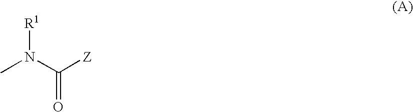
wherein R¹ is hydrogen or methyl;
Z is piperidine optionally substituted with methyl, cyclopentane substituted by amine or C(R²)(R³)N(R⁴)(R⁵);
R² and R³ are independently selected from hydrogen, methyl, ethyl, fluoromethyl and hydroxymethyl; and
R⁴ and R⁵ are independently selected from hydrogen, methyl, acetyl and N,N-dimethylaminomethylcarbonyl;
or R is a group of formula (B): 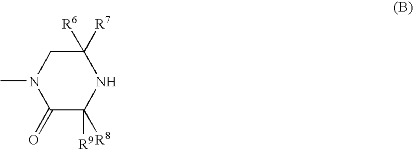
wherein R⁶⁻⁹ are independently selected from hydrogen and methyl and at least one of them is methyl.

2. A compound according to claim 1, wherein
R^{a} is aryl substituted by heteroaryl;
X is CH;
Y is a single bond;
R^{e} is hydrogen;
R^{f} is alkoxy;
R is a group of formula (A): 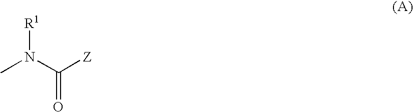
wherein
R¹ is hydrogen or methyl;
Z is C(R²)(R³)N(R⁴)(R⁵);
R² and R³ are independently selected from hydrogen and methyl;
R⁴ and R⁵ are independently selected from hydrogen or methyl; or
R is a group of formula (B): 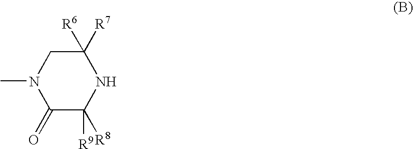
wherein R⁶ and R⁷ are hydrogen and R⁸ and R⁹ are methyl.

3. A compound according to claim 1, wherein
R^{a} is phenyl substituted by methyl-furanyl;
X is CH;
Y is a single bond;
R^{e} is hydrogen;
R^{f} is methoxy;
R is a group of formula (A): 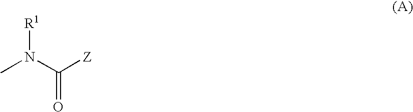
wherein
R¹ is hydrogen or methyl;
Z is C(R²)(R³)N(R⁴)(R⁵);
R² and R³ are independently selected from hydrogen and methyl;
R⁴ and R⁵ are independently selected from hydrogen or methyl; or
R is a group of formula (B): 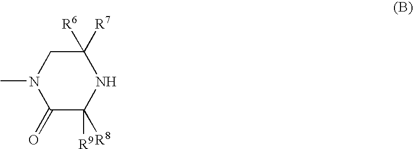
wherein R⁶ and R⁷ are hydrogen and R⁸ and R⁹ are methyl.

4. A compound according to claim 1 selected from the following:
N¹-[3-({[3-Fluoro-4-(5-methyl-2-furanyl)phenyl]sulfonyl}amino)-4-(methyloxy)phenyl]-2-methylalaninamide
N¹-[5-(3,3-Dimethyl-2-oxo-1-piperazinyl)-2-(methyloxy)phenyl]-3-fluoro-4-(5-methyl-2-furanyl)benzenesulfonamide
N¹-[3-({[3-Fluoro-4-(5-methyl-2-furanyl)phenyl]sulfonyl}amino)-4-(methyloxy)phenyl]-D-alaninamide
N¹-[3-({[3-Fluoro-4-(5-methyl-2-furanyl)phenyl]sulfonyl}amino)-4-(methyloxy)phenyl]-L-alaninamide h
N¹-[3-({[3-Fluoro-4-(5-methyl-2-furanyl)phenyl]sulfonyl}amino)-4-(methyloxy)phenyl]-glycinamide
N¹-[3-({[3-Fluoro-4-(5-methyl-2-furanyl)phenyl]sulfonyl}amino)-4-(methyloxy)phenyl]-N¹,2-dimethylalaninamide
(2S)-2-Amino-N-[3-({[3-fluoro-4-(5-methyl-2-furanyl)phenyl]sulfonyl}amino)-4-(methyloxy)phenyl]butanamide
N¹-[3-({[3-Fluoro-4-(5-methyl-2-furanyl)phenyl]sulfonyl}amino)-4-(methyloxy)phenyl]-L-serinamide
N¹-[3-({[3-Fluoro-4-(5-methyl-2-furanyl)phenyl]sulfonyl}amino)-4-(methyloxy)phenyl]-2-methylserinamide
N¹-[3-({[3-Fluoro-4-(5-methyl-2-furanyl)phenyl]sulfonyl}amino)-4-(methyloxy)phenyl]-N²-methyl-L-alaninamide
N¹-[3-({[3-Fluoro-4-(5-methyl-2-furanyl)phenyl]sulfonyl}amino)-4-(methyloxy)phenyl]-N²,2-dimethylalaninamide h
N¹-[3-({[2-Chloro-4-(5-methyl-2-furanyl)phenyl]sulfonyl}amino)-4-(methyloxy)phenyl]-2-methylalaninamide
2-Methyl-N¹-[3-({[4-(5-methyl-2-furanyl)phenyl]sulfonyl}amino)-4-(methyloxy)phenyl]alaninamide
N¹-[3-({[2-Chloro-4-(3-furanyl)phenyl]sulfonyl}amino)-4-(methyloxy)phenyl]-2-methylalaninamide
N¹-[3-({[3-Fluoro-4-(3-furanyl)phenyl]sulfonyl}amino)-4-(methyloxy)phenyl]-2-methylalaninamide
N¹-[3-({[2-Chloro-4-(5-methyl-2-furanyl)phenyl]sulfonyl}amino)-4-(methyloxy)phenyl]-N²-methyl-L-alaninamide
N¹-[3-({[4-(5-Methyl-2-furanyl)phenyl]sulfonyl}amino)-4-(methyloxy)phenyl]-D-alaninamide
N¹-[3-({[2-Chloro-4-(5-methyl-2-furanyl)phenyl]sulfonyl}amino)-4-(methyloxy)phenyl]-D-alaninamide
N¹-[3-({[2-Chloro-4-(5-methyl-2-furanyl)phenyl]sulfonyl}amino)-4-(methyloxy)phenyl]-L-alaninamide
N¹-[3-({[2-Chloro-4-(4-methyl-2-thienyl)phenyl]sulfonyl}amino)-4-(methyloxy)phenyl]-N²-methyl-L-alaninamide
N¹-[3-({[3-Chloro-3′-(methyloxy)-4-biphenylyl]sulfonyl}amino)-4-(methyloxy)phenyl]-N²-methyl-L-alaninamide
N¹-[3-({[2′-Fluoro-5′-(methyloxy)-4-biphenylyl]sulfonyl}amino)-4-(methyloxy)phenyl]-N²-methyl-L-alaninamide
N¹-[3-({[3-Chloro-2′-fluoro-5′-(methyloxy)-4-biphenylyl]sulfonyl}amino)-4-(methyloxy)phenyl]-N²-methyl-L-alaninamide
N¹-[3-({[2-Chloro-4-(5-methyl-2-furanyl)phenyl]sulfonyl}amino)-4-(methyloxy)phenyl]-N²-methyl-D-alaninamide
N¹-[5-({[3-Fluoro-4-(5-methyl-2-furanyl)phenyl]sulfonyl}amino)-2-(methyloxy)phenyl]-2-methylalaninamide
N¹-[3-({[3-Fluoro-4-(5-methyl-2-furanyl)phenyl]sulfonyl}amino)phenyl]-2-methylalaninamide
N¹-[4-Chloro-3-({[3-fluoro-4-(5-methyl-2-furanyl)phenyl]sulfonyl}amino)phenyl]-2-methylalaninamide
N¹-[4-Fluoro-3-({[3-fluoro-4-(5-methyl-2-furanyl)phenyl]sulfonyl}amino)phenyl]-2-methylalaninamide and
N¹-[6-({[3-Fluoro-4-(5-methyl-2-furanyl)phenyl]sulfonyl}amino)-5-(methyloxy)-2-pyridinyl]-2-methylalaninamide.

5. (canceled)

6. The method of treatment of claim 11, wherein the condition or disorder is selected from the group consisting of cachexia, sarcopenia, osteoporosis, rheumatoid arthritis, osteoarthritis, frailty associated with aging, growth hormone deficiency, metabolic disorders, sleep disorder, congestive heart failure, symptoms associated with gastro-esophageal reflux with or without dyspepsia and with or without appetite-/metabolic-related cachexia, treatments of paralytic ileus or pseudo-obstruction or conditions associated with constipation, and constipation-predominant irritable bowel syndrome.

7. (canceled)

8. (canceled)

9. A pharmaceutical composition comprising a compound according to claim 1 and a pharmaceutically acceptable carrier or excipient.

10. (canceled)

11. A method of treatment for a condition or a disorder in a human which can be mediated via the growth hormone secretagogue (GHS) receptors, which method comprises administering to said human a therapeutically effective amount of a compound of formula (I) or a pharmaceutically acceptable salt thereof.
